(19) United States
(12) Patent Application Publication
(10) Pub. No.: US 2004/0076975 A1
(43) Pub. Date: Apr. 22, 2004

(54) Methods for assessing the risk of non-insulin-dependent diabetes mellitus based on allelic variations in the 5'-flanking region of the insulin gene and body fat

(76) Inventor: Pierre Bougneres, Paris (FR)

Correspondence Address: BOZICEVIC, FIELD & FRANCIS LLP, 200 MIDDLEFIELD RD, SUITE 200, MENLO PARK, CA, 94025, US

(21) Appl. No.: 10/415,228

(22) PCT Filed: Oct. 31, 2001
(86) PCT No.: PCT/IB01/02747

(51) Int. Cl. C12Q 1/68

(52) U.S. Cl. 435/6

(57) ABSTRACT

The invention features methods for determining the risk of development of non-insulin dependent diabetes mellitus (NIDDM or type II diabetes) in a subject by examining both the insulin HphI locus and the body fat value of the patient. In related aspects, the invention features methods for diagnosing a subtype of NIDDM, as well as methods to facilitate rationale therapy and maintenance of NIDDM patients.

The present invention relates to methods of diagnosis and prognosis of diabetes and to methods of establishing inclusion criteria for clinical studies.

### BACKGROUND OF THE INVENTION

Diabetes Mellitus is a serious disease afflicting over 100 million people worldwide. In the United States, there are more than 12 million diabetics, with 600,000 new cases diagnosed each year.

### FIELD OF THE INVENTION

Diabetes mellitus is a diagnostic term for a group of disorders characterized by abnormal glucose homeostasis resulting in elevated blood sugar. There are many types of diabetes, but the two most common are Type I (also called insulin-dependent diabetes mellitus or IDDM) and Type II (also called non-insulin-dependent diabetes mellitus or NIDDM).

The etiology of the different types of diabetes are not the same; however, everyone with diabetes has two things in common: overproduction of glucose by the liver and little or no ability to move glucose out of the blood into the cells where it becomes the body's primary fuel.

People who do not have diabetes rely on insulin, a hormone made in the pancreas, to move glucose from the blood into the body's billions of cells. However, people who have diabetes either don't produce insulin or can't efficiently use the insulin they produce, therefore, they can't move glucose into their cells. Glucose accumulates in the blood creating a condition called hyperglycemia, and over time, can cause very serious health problems.

Diabetes is a syndrome with interrelated metabolic, vascular, and neuropathic components. The metabolic syndrome, generally characterized by hyperglycemia, comprises alterations in carbohydrate, fat and protein metabolism caused by absent or markedly reduced insulin secretion and/or ineffective insulin action. The vascular syndrome consists of abnormalities in the blood vessels leading to cardiovascular, retinal and renal complications. Abnormalities in the peripheral and autonomic nervous systems are also part of the diabetic syndrome.

People with IDDM , which accounts for about 5% to 10% of those who have diabetes, don't produce insulin and therefore must inject insulin to keep their blood glucose levels normal. IDDM is characterized by low or undetectable levels of endogenous insulin production caused by destruction of the insulin-producing β cells of the pancreas, the characteristic that most readily distinguishes IDDM from NIDDM. IDDM, once termed juvenile-onset diabetes, strikes young and older adults alik .

Ninety percent to 95% of people with diabetes have type II (or NIDDM). NIDDM subjects produce insulin, but the cells in their bodies are insulin resistant: the cells don't respond properly to the hormone, so glucose accumulates in their blood. NIDDM is characterized by a relative disparity between endogenous insulin production and insulin requirements, leading to elevated blood glucose levels. In contrast to IDDM, there is always some endogenous insulin production in NIDDM; many NIDDM patients have normal or even elevated blood insulin levels, while other NIDDM patients have inadequate insulin production Rotwein, P. et al. N Engl J Med. 308, 65-71 (1983). Most people diagnosed with NIDDM are age 30 or older, and half of all new cases are age 55 and older. Compared with whites and Asians, NIDDM is more common among Native Americans, African-Americans, Latinos, and Hispanics. In addition, the onset can be insidious, or even clinically inapparent, making diagnosis difficult

The primary pathogenic lesion on NIDDM has remained elusive. Many have suggested that primary insulin resistance of the peripheral tissues is the initial event. Genetic epidemiological studies have supported this view. Similarly, insulin secretion abnormalities have been argued as the primary defect in NIDDM. It is likely that both phenomena are important in the development of NIDDM, and genetic defects predisposing to both are likely to be important contributors to the disease process (Rimoin, D. L., et al. Emery and Rimoin's Principles and Practice of Medical Genetics 3^{rd} Ed. 1: 1401-1402 (1996).

Although the evidence from studies of familial aggregation and twins leaves no doubt as to the importance of genetic factors in the etiology of diabetes, there is little agreement as to the nature of the genetic factors involved. This confusion can largely be attributed to the genetic heterogeneity that is now known to exist in diabetes.

A number of candidate genes, including the insulin gene, the insulin receptor gene and the insulin-sensitive glucose transporter (GLUT 4) gene, have been tested for possible roles in the etiology of NIDDM, with mostly conflicting results. Early studies identified a restriction-fragment length polymorphism (RFLP) in the 5′-flanking region of the insulin gene on the short arm of human chromosome 11. The region begins approximately 500 base pairs before the insulin mRNA transcription start site, and thus appears to modulate expression of the insulin gene. The polymorphisms are generated by a variable number of tandemly repeated (VNTR) sequences. In Caucasians the VNTRs can be divided into class I (sized 0-600 bp) and class III (sized 1600-2400 bp) alleles (Bell G. I. et al. Proc Natl Acad Sci USA 1981;78:5759-63). These alleles are easily identifiable through use of RFLP analysis: the ‘+’ alleles (T) of the HphI locus (‘+’ indicating the site was cut by a restriction enzyme) are in complete linkage disequilibrium with class I alleles of the neighboring insulin VNTR, and ‘−’ alleles are in complete linkage disequilibrium with the class III alleles. Class I alleles and class III alleles are also referred to as ‘L’ alleles and ‘U’ alleles, respectively by Owerbach D., Poulsen, S., et al. Lancet. 1:880-883 (1982). In addition, 19 polymorphisms in chromosome 11p15.5 have been identified (Lucassen, A. M. et al. Nature Genet. 4, 305-310 (1993)), the disclosure of which is incorporated herein by reference in their entireties. This genomic region includes the tyrosine hydroxylase (TH), insulin like growth factor II (IGG2) and insulin genes.

Owerbach D., et al. (Lancet. 1:880-883 (1982)) conducted restriction fragment length polymorphism (RFLP) analysis in the 5′-flanking region of the insulin gene in 53 members of a large family, six of which were diabetic. An association was found between the larger (class III) allele and increased fasting glucose levels and decreased insulin response with increasing age. Therefore, Owerbach D., et al. concluded the larger alleles are genetic markers of NIDDM susceptibility. Similarly, Rotwein, P. et al. (N Engl J Med. 308:65-71 (1983)) found the long insertion more often in NIDDM subjects than in non-diabetics, and also concluded polymorphisms (length variations) in the 5′-flanking region of the insulin gene may provided a genetic marker for NIDDM. However, Permutt, A., et al. (Diabetes. 34:311-314 (1985)) conducted a similar experiment and found there was no differences in fasting insulin, glucose concentration or insulin secretory response to insulin gene polymorphic status in non-diabetic and NIDDM subjects. Thus, the results of the studies attempting to link the allelic variation in the 5′-flanking region of the insulin gene with NIDDM were contradictory and inconclusive.

Many people with NIDDM have sedentary lifestyles and are obese; they weigh at least 20% more than the recommended weight for their height and build. Further, obesity is characterized by hyperinsulinemia and insulin resistance, a feature shared with NIDDM, hypertension and atherosclerosis. In order to investigate the molecular genetics of upper body obesity (central) and hyperinsulinemia, Weaver, J. U., et al. (Eur J Clin Invest. 22:265-270 (1992)) examined 56 severely obese, non-diabetic women for association of insulin gene RFLPs with anthropometric measurements and indices of insulin secretion and resistance. Weaver, J. U., et al. found that class III alleles were associated with central obesity, fasting hyperinsulinemia, stimulated insulin secretion and insulin resistance. Therefore, they concluded that polymorphisms in the 5′-flanking region of the insulin gene may affect expression of the gene and thereby modulate insulin production in severely obese female subjects. However, in a study that analyzed the insulin VNTR in 218 men with low birth weight and NIDDM, Ong, K. K. L. et al. (Nature Genet. 21:262-263 (1999)), found the insulin VNTR and birth weight have independent effects on risk for NIDDM. Again, results attempting to answer the relationship between obesity and NIDDM were conflicting and a new means of investigating possible genetic components of diabetes were necessary.

Perhaps the most problematic aspect of studying the genetics of NIDDM is the likely extensive etiologic heterogeneity which underlies this disease. Generic defects likely influence any of the many steps involved in glucose regulation. Each of these defects, either alone or in concert with other defects, could result in NIDDM. While such etiologic complexity by no means precludes genetic investigations, extensive etiologic heterogeneity implies that to understand particular pathogenetic mechanisms, one must be able to measure physiologic “defects” at a more specific level than the gross phenotype of glucose intolerance (Raffel et al. Emery and Rimoin's Principles and Practice of Medical Genetics. 3^{rd} ed. 1421 (1996)). One such example of a measurable physiologic defect or precursor is obesity, as discussed herein.

Obesity and diabetes are among the most common human health problems in industrialized societies. In industrialized countries a third of the population is at least 20% overweight. In the United States, the percentage of obese people has increased from 25% at the end of the 70s, to 33% at the beginning of the 90's. Obesity is one of the most important risk factors for NIDDM. Definitions of obesity differ, but in general, a subject weighing at least 20% more than the recommended weight for his or her height and build is considered obese. The risk of developing NIDDM is tripled in subjects 30% overweight, and three-quarters of people with NIDDM are overweight.

Obesity, which is the result of an imbalance between caloric intake and energy expenditure, is highly correlated with insulin resistance and diabetes in experimental animals and humans. However, the molecular mechanisms that are involved in obesity-diabetes syndromes are not clear. During early development of obesity, increased insulin secretion balances insulin resistance and protects patients from hyperglycemia (Le Stunff, et al., Diabetes.43, 696-702 (1994)). However, after several decades, β cell function deteriorates and non-insulin-dependent diabetes develops in about 20% of the obese population (Pedersen, P. Diab. Metab. Rev. 5, 505-509 (1989)) and (Brancati, F. L., et al., Arch Intern Med. 159, 957-963 (1999)). Given its high prevalence in modern societies, obesity has thus become the leading risk factor for NIDDM (Hill, J. O., et al., Science. 280, 1371-1374 (1998)). However, the factors which predispose a fraction of patients to alterations of insulin secretion in response to fat accumulation remain unknown.

Obesity considerably increases the risk of developing cardiovascular diseases as well. Coronary insufficiency, atheromatous disease, and cardiac insufficiency are at the forefront of the cardiovascular complications induced by obesity. It is estimated that if the entire population had an ideal weight, the risk of coronary insufficiency would decrease by 25%, and the risk of cardiac insufficiency and of cerebral vascular accidents by 35%. The incidence of coronary diseases is doubled in subjects under 50 years who are 30% overweight The diabetic patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of obesity (Perry, I. J. et al. BMJ 310, 560-564 (1995)).

Diabetes has also been implicated in the development of kidney disease, eye diseases and nervous-system problems. Kidney disease, also called nephropathy, occurs when the kidney's “filter mechanism” is damaged and protein leaks into urine in excessiv amounts and eventually the kidney fails. Diabetes is also a leading cause of damage to the retina at the back of the eye and increases risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

Currently, diabetes can't be cured, but the disease can be managed. Existing treatments for NIDDM, which has not changed substantially in many years, are all with limitations. While physical exercise and reductions in dietary intake of calories will dramatically improve the diabetic condition, compliance with this treatment is very poor because of well-entrenched sedentary lifestyles and excess food consumption, especially high fat-containing food. Increasing the plasma level of insulin by administration of sulfonylureas (e.g. tolbutamide, glipizide) which stimulate the pancreatic β-cells to secrete more insulin or by injection of insulin after the response to sulfonylureas fails, will result sufficient insulin concentrations to stimulate the very insulin-resistant tissues. However, dangerously low levels of plasma glucose can result from these last two treatments, increasing insulin resistance due to the even higher plasma insulin levels could also theoretically occur. The biguanides increase insulin sensitivity resulting in some correction of hyperglycemia. However, the two biguanides, phenformin and metformin, can induce lactic acidosis and nausea/diarrhea, respectively.

### SUMMARY OF THE INVENTION

The invention features methods for determining the risk of development of non-insulin dependent diabetes mellites (NIDDM or type II diabetes) in a subject by examining both the insulin HphI locus and the body fat value of the patient. In related aspects, the invention features methods for diagnosing a subtype of NIDDM, as well as methods to facilitate rationale therapy and maintenance of NIDDM patients.

The invention results from the discovery that homozygotes of the HphI locus of the insulin gene along with body fat measurement serve as an excellent indicator of NIDDM susceptibility. The inventor investigated the influence of HphI genotypes on the relationship between obesity and insulin levels in obese juveniles, and found HphI [+/+] homozygotes (insulin VNIR I/I) showed a stronger correlation between insulin and BMI than those with HphI [+/−] or [−/−] genotypes (insulin VNTR I/III and insulin VNTR III/III, respectively) and a comparable adiposity. Therefore, obese individuals with HphI [+/−] or [−/−] genotypes are significantly more likely to develop NIDDM than obese individuals with HphI [+/+] genotypes.

In a first embodiment, the invention features a method of determining the risk of developing NIDDM in an individual, comprising: a) determining the identity of the polymorphic base(s) of at least one marker in linkage disequilibrium with the insulin HphI locus of the individual; b) determining a body fat value for the individual; and c) assigning a risk value based on said marker identity, said body fat value and a predetermined value that correlates said identity, said body fat value and said risk of developing NIDDM. In another aspect, the invention features a method of determining the risk of developing NIDDM in an individual, comprising: a) determining the VNTR class of an insulin gene of the individual; b) determining a body fat value for the individual; and c) assigning a risk value based on said VNTR class, said body fat value and a predetermined value that correlates said VNTR class, said body fat value and said risk of developing NIDDM. In yet another aspect, the invention features a method of determining the risk of developing NIDDM in an individual, comprising: a) genotyping a marker in linkage disequilibrium with the insulin HphI locus by determining the identity of the nucleotides at said marker for both copies of said marker present in the genome of an individual; b) genotyping a second marker by determining the identity of the nucleotides at said second genetic marker for both copies of said second marker present in the genome of the individual; c) determining a body fat value for said individual; and d) assigning a risk value based on said identities of steps a) and b), said body fat value of step c) and a predetermined value that correlates said identity, said body fat value and said risk of developing NIDDM. In addition, the methods of determining the risk of developing NIDDM in an individual encompass methods with any further limitation described in this disclosure, or those following, specified alone or in any combination: Optionally, said identity of the polymorphic base(s) at said marker is determined for both copies of said marker present in said individual's genome; Optionally, said VNTR class of the insulin gene is determined for both copies of said VNTR present in said individual's genome; Optionally, said second marker is in linkage disequilibrium with the insulin HphI locus. Optionally, said marker in linkage disequilibrium with the insulin HphI locus may be selected from the markers provided in Table C; preferably markers −4217 PstI, −2221 MspI, −23 HphI, +1428 FokI, +11000 AluI and +32000 ApaI; or more preferably marker −23 HphI. Optionally, said marker in linkage disequilibrium with the insulin HphI locus may further include any other marker that is in linkage disequilibrium with the insulin HphI locus that is known in the art; as well as any marker determined to be in linkage disequilibrium with the insulin HphI locus by methods described herein.

In a second embodiment, the invention features a method of diagnosing a subtype of NIDDM in an individual, comprising: a) determining the identity of the polymorphic base(s) of at least one marker in linkage disequilibrium with the insulin HphI locus of the individual; b) determining a body fat value for the individual; and c) assigning a subtype based on said marker identity, said body fat value and a predetermined value that correlates said identity, said body fat value and likelihood of having a particular subtype of NIDDM. In another aspect, the invention features a method of diagnosing a subtype of NIDDM in an individual, comprising: a) determining the VNTR class of an insulin gene of the individual; b) determining a body fat value for the individual; and c) assigning a subtype based on said VNTR class, said body fat value and a predetermined value that correlates said VNTR class, said body fat value and likelihood of having a particular subtype of NIDDM. In yet another aspect, the invention features a method of diagnosing a subtype of NIDDM in an individual, comprising: a) genotyping a marker in linkage disequilibrium with the insulin HphI locus by determining the identity of the nucleotides at said marker for both copies of said marker present in the genome of each individual; b) genotyping a second marker by determining the identity of the nucleotides at said second genetic marker for both copies of said second marker present in the genome of the individual; c) determining a body fat value for said individual; and d) assigning a subtype based on said identities of steps a) and b), said body fat value of step c) and a predetermined value that correlates said identity, said body fat value and likelihood of having a particular subtype of NIDDM. In addition, the methods of diagnosing a subtype of NIDDM in an individual encompass methods with any further limitation described in this disclosure, or those following, specified alone or in any combination: Optionally, said identity of the polymorphic base(s) at said marker is determined for both copies of said marker present in said individual's genome; Optionally, said VNTR class of the insulin gene is determined for both copies of said VNTR present in said individual's genome; Optionally, said second marker is in linkage disequilibrium with the insulin HphI locus. Optionally, said marker in linkage disequilibrium with the insulin HphI locus may be selected from the markers provided in Table C; preferably markers −4217 PstI, −2221 MspI, −23 HphI, +1428 FokI, +11000 AluI and +32000 ApaI; or more preferably marker −23 HphI. Optionally, said marker in linkage disequilibrium with the insulin HphI locus may further include any other marker that is in linkage disequilibrium with the insulin HphI locus that is known in the art; as well as any marker determined to be in linkage disequilibrium with the insulin HphI locus by methods described herein.

In a third embodiment, the invention features a method of treatment or prophylaxis of NIDDM for an individual comprising a method of prognosis of the invention and administering a weight loss regime, wherein said weight loss regime is selected from the group consisting of food restriction, increased calorie use, gastrointestinal surgery, medicinal approaches and reduced absorption of dietary lipids. In addition, the methods of treatment or prophylaxis of NIDDM for an individual encompass methods with any further limitation described in this disclosure, or those following, specified alone or in any combination

In a fourth embodiment, the invention features a method for selecting in a clinical or association study of an insulin-related disorder, comprising: a) determining the identity of the polymorphic base(s) of at least one marker in linkage disequilibrium with the insulin HphI locus of the individual; b) determining a body fat value for the individual; and c) including the individual based on said marker identity, said body fat value and a predetermined value that correlates said identity, said body fat value and likelihood of having an insulin-related disorder. In another aspect, the invention features a method for selecting an individual for a clinical or association study of an insulin-related disorder comprising: a) determining the VNTR class of an insulin gene of the individual; b) determining a body fat value for the individual; and c) including the individual in the study based on said VNTR class, said body fat value and a predetermined value that correlates said VNTR class, said body fat value and likelihood of having an insulin-related disorder. In yet another aspect, the invention features a method of identifying an individual for a clinical or association study of an insulin-related disorder comprising: a) genotyping a marker in linkage disequilibrium with the insulin HphI locus by determining the identity of the nucleotides at said marker for both copies of said marker present in the genome of each individual; b) genotyping a second marker by determining the identity of the nucleotides at said second genetic marker for both copies of said second marker present in the genome of the individual; c) determining a body fat value for said individual; and d) including the individual in the study based on said identities of steps a) and b), said body fat value of step c) and a predetermined value that correlates said identity, said body fat value and likelihood of having an insulin-related disorder. In addition, the methods of this embodiment encompass methods with any further limitation described in this disclosure, or those following, specified alone or in any combination: Optionally, said identity of the polymorphic base(s) at said marker is determined for both copies of said marker present in said individual's genome; Optionally, said VNTR class of the insulin gene is determined for both copies of said VNTR present in said individual's genome; Optionally, said second marker is in linkage disequilibrium with the insulin HphI locus. Optionally, said marker in linkage disequilibrium with the insulin HphI locus may be selected from the markers provided in Table C; preferably markers −4217 PstI, −2221 MspI, −23 HphI, +1428 FokI, +11000 AluI and +32000 ApaI; or more preferably marker −23 HphI. Optionally, said marker in linkage disequilibrium with the insulin HphI locus may further include any other marker that is in linkage disequilibrium with the insulin HphI locus that is known in the art; as well as any marker determined to be in linkage disequilibrium with the insulin HphI locus by methods described herein

In a fifth embodiment, the invention encompasses methods of estimating the frequency of a haplotype for a set of genetic markers in a population suffering from juvenile obesity, comprising: a) genotyping a marker in linkage disequilibrium with the insulin HphI locus by determining the identity of the nucleotides at said marker for both copies of said marker present in the genome of each individual in said population; b) genotyping a second marker by determining the identity of the nucleotides at said second genetic marker for both copies of said second marker present in the genome of each individual in said population; and c) applying a haplotype determination method to the identities of the nucleotides determined in steps a) and b) to obtain an estimate of said frequency. In addition, the methods of estimating the frequency of a haplotype of the invention encompass methods with any flitter limitation described in this disclosure, or those following, specified alone or in any combination: Optionally said haplotype determination method is selected from the group consisting of asymmetric PCR amplification, double PCR amplification of specific alleles, the Clark method, or an expectation maximization algorithm; Optionally, said second marker is in linkage disequilibrium with the insulin HphI locus. Optionally, said marker in linkage disequilibrium with the insulin HphI locus may be selected from the markers provided in Table C; preferably markers −4217 PstI, −2221 MspI, −23 HphI, +1428 FokI, +11000 AluI and +32000 ApaI; or more preferably marker −23 HphI. Optionally, said marker in linkage disequilibrium with the insulin HphI locus may further include any other marker that is in linkage disequilibrium with the insulin HphI locus that is known in the art; as well as any marker determined to be in linkage disequilibrium with the insulin HphI locus by methods described herein.

In a sixth embodiment, the invention encompasses methods of detecting an association between a haplotype and an insulin-related disorder, comprising: a) estimating the frequency of at least one haplotype in a population suffering from said insulin-related disorder according to the method of estimating the frequency of a haplotype of the invention; b) estimating the frequency of said haplotype in a control population according to the method of estimating the frequency of a haplotype of the invention; and c) determining whether a statistically significant association exists between said haplotype and said insulin-related disorder. In addition, the methods of detecting an association between a haplotype and a trait of the invention encompass methods with any further limitation described in this disclosure, or those following, specified alone or in any combination: Optionally, said insulin-related disorder is hyperinsulinemia or a predisposition to hyperinsulinemia. Optionally, said haplotype consists of markers in linkage disequilibrium with the insulin HphI locus which be selected from the markers provided in Table C; preferably markers −4217 PstI, −2221 MspI, −23 HphI, +1428 FokI, +11000 AluI and +32000 ApaI; or more preferably marker −23 HphI. Optionally, said haplotype consists of markers in linkage disequilibrium with the insulin HphI locus which may further include any other marker that is in linkage disequilibrium with the insulin HphI locus that is known in the art; as well as any marker determined to be in linkage disequilibrium with the insulin HphI locus by methods described herein.

### BRIEF DESCRIPTION OF THE DRAWINGS

FIG. 1 is a diagrammatic representation of the TH-INS-IGF2 region on chromosome 11p15.5 showing the positions of polymorphisms in genomic DNA. Open boxes refer to introns; closed boxes to exons; and hatched boxes to untranslated regions. The triangle depicts the INS VNTR locus. Polymorphisms are designated by their position with respect to the first base of the initiating ATG (+1) codon of INS.

FIG. 2A consists of two graphs that demonstrate the relationship between fasting plasma insulin and fatness in the 458 obese children of GenOb cohort I with respect to their Hph1 genotype.

FIG. 2B consists of two graphs that demonstrate the relationship between fasting plasma insulin and body mass index in the obese boys in the two Hph1 (insulin VNTR) genotype homozygous subgroups.

FIG. 3 is a graph that shows the averaged longitudinal weight curves (normalized to the normal weight value for age and height) versus chronological age in the two genotypic groups of obese children. The curves were constructed from the study of a subset of 332 patients whose yearly individual data could be collected from Health Personal Bulletins, starting at birth until time of study.

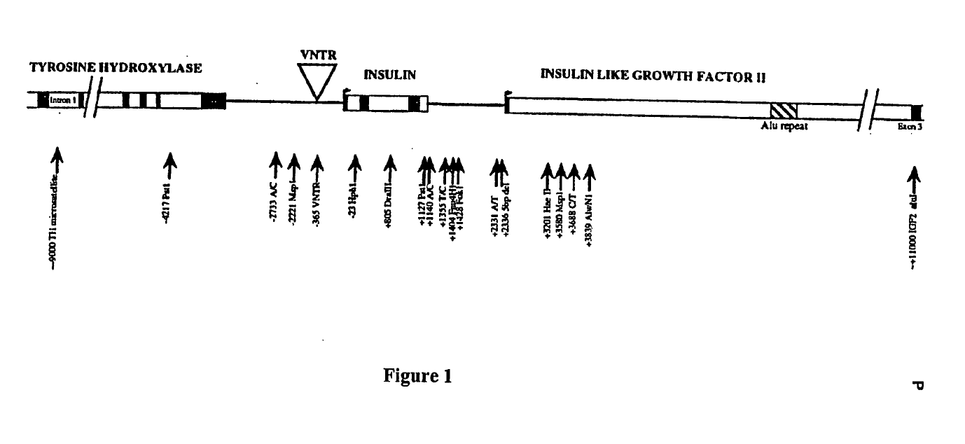

### DETAILED DESCRIPTION OF THE INVENION

Before the present invention is described, it is to be understood that this invention is not limited to the particular embodiments described, as such may, of course, vary. It is also to be understood that the terminology used herein is for the purpose of describing particular embodiments only, and is not intended to limit the scope of the present invention, which will be limited only to the appended claims.

It must be noted that as used herein and in the appended claims, the singular forms “a”, “an,” and “the” include plural referents unless the context clearly dictates otherwise. Thus, for example, reference to “an individual” includes one or more individuals, and reference to “the method” includes reference to equivalent steps and methods known to those skilled in the art, and so forth.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned herein are incorporated by reference to disclose and describe the specific methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

Before describing the invention in greater detail, the following definitions are set forth to illustrate and define the meaning and scope of the terms used to describe the invention herein.

The terms “insulin gene,” when used herein, encompasses genomic, mRNA and cDNA sequences encoding the polypeptide hormone insulin, including the untranslated regulatory regions of the genomic DNA

The term “isolated” requires that the material be removed from its original environment (e. g., the natural environment if it is naturally occurring). For example, a naturally-occurring polynucleotide or polypeptide present in a living animal is not isolated, but the same polynucl otide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “isolated” further requires that the material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated. Specifically excluded from the definition of “isolated” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified polynucleotide of the present invention makes up less than 5% of the number of nucleic acid inserts in the vector molecules. Further specifically excluded are whole cell genomic DNA or whole cell RNA preparations (including said whole cell preparations which are mechanically sheared or enzymaticly digested). Further specifically excluded are the above whole cell preparations as either an in vitro preparation or as a heterogeneous mixture separated by electrophoresis (including blot transfers of the same) wherein the polynucleotide of the invention has not further been separated from the heterologous polynucleotides in the electrophoresis medium (e.g., further separating by excising a single band from a heterogeneous band population in an agarose gel or nylon blot).

The term “purified” does not require absolute purity; rather, it is intended as a relative definition. Purification of starting material or natural material to at least one order of magnitude, preferably two or three orders, and more preferably four or five orders of magnitude is expressly contemplated. As an example, purification from 0.1% concentration to 10% concentration is two orders of magnitude. The term “purified polynucleotide” is used herein to describe a polynucleotide or polynucleotide vector of the invention which has been separated from other compounds including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially pure when at least about 50%, preferably 60 to 75% of a sample exhibits a single polynucleotide sequence and conformation (linear versus covalently close). A substantially pure polynucleotide typically comprises about 50%, preferably 60 to 90% weight/weight of a nucleic acid sample, more usually about 95%, and preferably is over about 99% pure. Polynucleotide purity or homogeneity is indicated by a number of means well known in the art, such as agarose or polyacrylamide gel electrophoresis of a sample, followed by visualizing a single polynucleotide band upon staining the gel. For certain purposes higher resolution can be provided by using HPLC or other means well known in the art

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

The term “purified polypeptide” is used herein to describe a polypeptide of the invention which has been separated from other compounds including, but not limited to nucleic acids, lipids, carbohydrates and other proteins. A polypeptide is substantially pure when at least about 50%, preferably 60 to 75% of a sample exhibits a single polypeptide sequence. A substantially pure polypeptide typically comprises about 50%, preferably 60 to 90% weight/weight of a protein sample, more usually about 95%, and preferably is over about 99% pure. Polypeptide purity or homogeneity is indicated by a number of means well known in the art, such as polyacrylamide gel electrophoresis of a sample, followed by visualizing a single polypeptide band upon staining the gel. For certain purposes higher resolution can be provided by using HPLC or other means well known in the art.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule.

As used interchangeably herein, the terms “nucleic acids”, “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064. The polynucleotide sequences of the invention may be prepared by any known method, including synthetic, recombinant, ex vivo generation, or a combination thereof, as well as utilizing any purification methods known in the art.

A “promoter” refers to a DNA sequence recognized by the synthetic machinery of the cell required to initiate the specific transcription of a gene.

A sequence which is “operably linked” to a regulatory sequence such as a promoter means that said regulatory element is in the correct location and orientation in relation to the nucleic acid to control RNA polymerase initiation and expression of the nucleic acid of interest.

As used herein, the term “operably linked” refers to a linkage of polynucleotide elements in a functional relationship. For instance, a promoter or enhancer is operably linked to a coding sequence if it affects the transcription of the coding sequence. More precisely, two DNA molecules (such as a polynucleotide containing a promoter region and a polynucleotide encoding a desired polypeptide or polynucleotide) are said to be “operably linked” if the nature of the linkage between the two polynucleotides does not (1) result in the introduction of a frame-shift mutation or (2) interfere with the ability of the polynucleotide containing the promoter to direct the transcription of the coding polynucleotide.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The terms “trait” and “phenotype” are used interchangeably herein and refer to any visible, detectable or otherwise measurable property of an organism such as symptoms of, or susceptibility to a disease for example. Typically the terms “trait” or “phenotype” are used herein to refer to symptoms of, or susceptibility to a disease, a beneficial response to or side effects related to a treatment Preferably, said trait can be, but not limited to, obesity related disorders and/or diabetes mellitus.

The term “allele” is used herein to refer to variants of a nucleotide sequence. A biallelic polymorphism has two forms. Diploid organisms may be homozygous or heterozygous for an allelic form.

The term “heterozygosity rate” is used herein to refer to the incidence of individuals in a population which are heterozygous at a particular allele. In a biallelic system, the heterozygosity rate is on average equal to 2Pₐ(1−Pₐ), where Pₐ is the frequency of the least common allele. In order to be useful in genetic studies, a genetic marker should have an adequate level of heterozygosity to allow a reasonable probability that a randomly selected person will be heterozygous.

The term “genotype” as used herein refers the identity of the alleles present in an individual or a sample. In the context of the present invention, a genotype preferably refers to the description of the genetic marker alleles present in an individual or a sample. The term “genotyping” a sample or an individual for a genetic marker involves determining the specific allele or the specific nucleotide carried by an individual at a genetic marker.

The term “mutation” as used herein refers to a difference in DNA sequence between or among different genomes or individuals which has a frequency below 1%.

The term “haplotype” refers to a combination of alleles present in an individual or a sample. In the context of the present invention, a haplotype preferably refers to a combination of genetic marker alleles found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is the replacement of one nucleotide by another nucleotide at the polymorphic site. Deletion of a single nucleotide or insertion of a single nucleotide also gives rise to single nucleotide polymorphisms. In the context of the present invention, “single nucleotide polymorphism” preferably refers to a single nucleotide substitution. Typically, between different individuals, the polymorphic site may be occupied by two different nucleotides.

The term “biallelic polymorphism” and “genetic marker” are used interchangeably herein to refer to a single nucleotide polymorphism having two alleles at a fairly high frequency in the population. A “genetic marker allele” refers to the nucleotide variants present at a genetic marker site. Typically, the frequency of the less common allele of the genetic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A genetic marker wherein the frequency of the less common allele is 30% or more is termed a “high quality genetic marker”.

The invention also concerns markers in linkage disequilibrium with the insulin HphI locus. The term “marker in linkage disequilibrium with the insulin HphI locus” is used herein to relate to the genetic markers described in Table C; preferably markers −4217 PstI, −2221 MspI, −23 HphI, +1428 FokI, +11000 AluI and +32000 ApaI; or more preferably marker −23 HphI. The term “marker in linkage disequilibrium with the insulin HphI locus” may include any other marker that is in linkage disequilibrium with the insulin HphI locus that is known in the art; as well as any marker determined to be in linkage disequilibrium with the insulin HphI locus by methods described herein.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within 1 nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within 1 nucleotide of the center” and any of the four nucleotides in the middle of the polynucleotide would be considered to be “within 2 nucleotides of the center”, and so on. For polymorphisms which involve the substitution, insertion or deletion of 1 or more nucleotides, the polymorphism, allele or genetic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4^{th} edition, 1995).

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind.

The term “insulin-related disorder” refers to any disorder known in the art in which insulin production, secretion or function (i.e., insulin resistance) is altered in an individual. The term “insulin-related disorder” particularly refers to insulin-dependent diabetes mellitus (IDDM or Type I diabetes), or non-insulin dependent diabetes mellitus (NIDDM or Type II diabetes), gestational diabetes, autoimmune diabetes, hyperinsulinemia, hyperglycemia, hypoglycemia, β-cell failure, insulin resistance, dyslipemias, atheroma and insulinoma. The term “insulin-related disorder” further refers to obesity and obesity related disorders such as obesity-related NIDDM, obesity-related atherosclerosis, heart disease, obesity-related insulin resistance, obesity-related hypertension, microangiopathic lesions resulting from obesity-related NIDDM, ocular lesions caused by microangiopathy in obese individuals with obesity-related NIDDM, and renal lesions caused by microangiopathy in obese individuals with obesity-related NIDDM.

The terms “agent acting on an insulin-related disorder” refers to a drug or a compound modulating the activity of insulin production, insulin secretion, insulin function, decreasing the body weight of obese individuals, or treating an insulin-related condition selected from the group consisting of IDDM, NIDDM, gestational diabetes, autoimmune diabetes, hyperinsulinemia, hyperglycemia, hypoglycemia, β-cell failure, insulin resistance, dyslipemias, atheroma, insulinoma, obesity and obesity related disorders as defined herein.

The terms “response to an agent acting on an insulin-related disorder” refer to drug efficacy, including but not limited to ability to metabolize a compound, to the ability to convert a pro-drug to an active drug, and to the pharmacokinetics (absorption, distribution, elimination) and the pharmacodynamics (receptor-related) of a drug in an individual.

The terms “side effects to an agent acting on an insulin-related disorder” refer to adverse effects of therapy resulting from extensions of the principal pharmacological action of the drug or to idiosyncratic adverse reactions resulting from an interaction of the drug with unique host factors.

The term “NIDDM” as used herein refers to non-insulin-dependent diabetes mellitus or Type II diabetes (the two terms are used interchangeably throughout this document). NIDDM refers to a condition in which there is a relative disparity between endogenous insulin production and insulin requirements, leading to an elevated blood glucose.

The term “weight loss regime” as used herein refers to any treatment known in the art aimed at reducing body mass. Weight loss regimes include food restriction, increased calorie use, gastrointestinal surgery, medicinal approaches and reduced absorption of dietary lipids.

The term “patient” as used herein refers to a mammal, including animals, preferably mice, rats, dogs, cattle, sheep, or primates, most preferably humans that are in need of treatment. The term “in need of such treatment” as used herein refers to a judgment made by a physician in the case of humans that a patient requires treatment. This judgment is made based on a variety of factors that are in the realm of a physician's expertise, but that include the knowledge that the patient is ill, or will be ill, as the result of a condition that is treatable by the compounds of the invention.

Similarly, the term “individual” as used herein refers to a mammal, including animals, preferably mice, rats, dogs, cattle, sheep, or primates, most preferably humans that perceives a need to reduce body mass (or that someone perceives the need to reduce body mass for). The term “perceives a need” refers to modulations (increases) in body mass that are typically below the cut-off for clinical obesity, although could also include clinical obesity. “Modulations in body mass” is defined above.

NIDDM and Obesity

Obesity, which is the result of an imbalance between caloric intake and energy expenditure, is highly correlated with insulin resistance and diabetes in experimental animals and humans. However, the molecular mechanisms that are involved in obesity-diabetes syndromes are not clear. During early development of obesity, increased insulin secretion balances insulin resistance and protects patients from hyperglycemia (Le Stunff; C., et al., Diabetes. 43, 696-702 (1994)). However, after several decades, β cell function deteriorates and an obesity-related subtype of NIDDM (gestational diabetes) develops in about 20% of the obese population (Pedersen, P. Diab. Metab. Rev. 5, 505-509 (1989)) and (Brancati, F. L., Wang, N. Y, Mead, L. A, Liang, K. Y, Klag, M. J. Arch Intern Med. 159, 957-963 (1999)) and (Arner, P. et al. Diabetologia. 34, 483-487 (1991). Given its high prevalence in modern societies, obesity has thus become the leading risk factor for NIDDM (Hill, J. O., et al., Science. 280, 1371-1374 (1998)).

The factors which predispose a fraction of patients to alterations of insulin secretion in response to fat accumulation remain unknown. To address this question, the inventor studied insulin levels within the dynamic phase of juvenile onset obesity. Studies of insulin secretion in adult patients may be exposed to biases: age-related differences, ethnicity, unknown obesity history, time-dependent β cell failure, changes due to diets and drugs, and varying glycemic status. However, obese children initially have normal fasting insulin as well as insulin sensitivity (Le Stunff, et al., Diabetes. 43, 696-702 (1994)) and develop hyperinsulinemia only after several years of obesity, allowing a more reliable study of the early β-cell response to obesity-related signals.

Obese patients were genotyped at the −23 HphI locus (polymorphisms are designated by their position with respect to the first base of the initiating ATG (+1) codon of the insulin gene), a polymorphism adjacent to the translational initiation codon of the insulin gene (Lucassen, A. M. et al. Nature Genet. 4, 305-310 (1993)). This RFLP is in strong linkage disequilibrium with the neighboring insulin VNTR: the ‘+’ alleles (T) of the HphI locus are in complete linkage disequilibrium with class I alleles of the neighboring insulin VNTR, and ‘−’ alleles (A) with the class III alleles. Therefore, the study tests the insulin VNTR through the −23 HphI polymorphism as a surrogate marker. Polymorphisms of the VNTR appear to modulate insulin gene transcription (Kennedy, C. G., et al., Nature Genet. 9, 293-298 (1995).

In young obese individuals, HphI allele and genotype frequencies were comparable to those in lean Caucasian subjects; however, HphI genotypes were associated with differences in fasting insulin levels. Patients with HphI [+/+] genotypes, although younger, showed higher insulin levels than those with HphI[+/−] or [−/−] genotypes and a comparable adiposity. The difference was more pronounced in super obese children whose fasting insulin levels are appreciatively 60-70% higher in HphI [+/+] individuals. In the whole obese cohort, plasma insulin and BMI were correlated (r=0.54, p<0.0001). Covariance analysis showed that HphI genotype had a major influence on the relationship between insulin and BMI (p<0.0001). HphI [+/+] homozygotes (insulin VNTR I/I) showed a stronger correlation between insulin and BMI than the two other genotypes. A highly significant association of the insulin level relation to BMI was also observed with the neighboring markers (−4217 PstI, −2221 MspI, +1428 FokI, +11000 AluI) that all are in strong LD with HphI alleles (Lucassen, A. M. et al. Nature Genet. 4, 305-310 (1993)).

Also, the inventor hypothesized heterozygous HphI [+/−] or [−/−], i.e. VNTR I/III and III/III, women could have a low insulin response to increased fatness during pregnancy, leading to increased glycaemia with potential effects on the size of their I/III or III/III fetuses. However, this hypothesis does not preclude the effect of the paternal alleles on conceptus birth weight

The inventor found an association between insulin genotypes and insulin levels involving the insulin VNTR, which is located only 360 bp from the HphI locus and is in almost complete linkage disequilibrium with its alleles (Bennett, S. T., et al., Annu Rev Genet. 30, 343-370 (1996). Moreover, its effect upon insulin gene transcription (Lucassen, A. M. et al. Hum Mol Genet. 4, 501-506 (1995)) makes the insulin VNTR a likely candidate to explain the results of the experiments. Based on observations made by the inventor, the HphI polymorphism and neighboring VNTR represent the first locus to be involved in the genetic regulation of fasting insulin levels, a trait whose heritability in humans is estimated between 0.20 and 0.52 (Snieder, H., et al., Genet Epidemiol. 16, 426-446 (1999)).

According to the data described herein, insulin VNTR and HphI polymorphisms are genetic markers for the failure of β-cell to cope with insulin resistance and NIDDM susceptibility in young obese patients. Thus, the invention features a method of determining the risk of developing NIDDM in an individual, comprising: genotyping at least one marker in linkage disequilibrium with the insulin HphI locus of the individual; determining a body fat value for the individual; and assigning a risk value based on the genotyping, the body fat value and a predetermined value that correlates genotype, body fat value and said risk of developing NIDDM.

In another aspect, the invention features a method of determining the risk of developing NIDDM in an individual as described above; however, the VNTR class of the insulin gene is determined and used to determine risk. For example, the risk of developing NIDDM is based on the VNTR class, the body fat value and a predetermined value that correlates the VNTR class, the body fat value and the risk of developing NIDDM, as described in below.

TABLE IA
Risk value of developing NIDDM

| | HAPLOTYPE | | |
|---|---|---|---|
| BMI (kg/m²) | [+/+] | [+/−] | [−/−] |
| 18.5 | * | * | * |
| 18.5-24.9 | * | * | * |
| 25.0-29.9 | * | ** | ** |
| 30.0-39.9 | * | *** | *** |
| >40.0 | ** | *** | *** |

*** indicates high risk of developing NIDDM
** indicates moderate risk of developing NIDDM
* indicates low risk of developing NIDDM

TABLE IB
Risk value of developing NIDDM

| | VNTR CLASS | | |
|---|---|---|---|
| BMI (kg/m²) | [I/I] | [I/III] | [III/III] |
| 18.5 | * | * | * |
| 18.5-24.9 | * | * | * |
| 25.0-29.9 | * | ** | ** |
| 30.0-39.9 | * | *** | *** |
| >40.0 | ** | *** | *** |

*** indicates high risk of developing NIDDM
** indicates moderate risk of developing NIDDM
* indicates low risk of developing NIDDM

Subtypes of NIDDM

Perhaps the most problematic aspect of studying the genetics of NIDDM is the likely extensive etiologic heterogeneity which underlies this disease. Generic defects influence any of the many steps involved in glucose regulation. Each of these defects, either alone or in concert with other defects, could result in NIDDM. While such etiologic complexity by no means precludes genetic investigations, extensive etiologic heterogeneity implies that to understand particular pathogenetic mechanisms, one must be able to measure physiologic “defects” at a more specific level than the gross phenotype of glucose intolerance (Raffel et al. Emery and Rimoin's Principles and Practice of Medical Genetics. 3^{rd} ed. 1421 (1996)).

In the present invention, the inventor studied insulin levels within the dynamic phase of juvenile onset obesity. It is known that increasing amounts of adipose tissue have a detrimental effect on whole-body sensitivity to the actions of insulin and glucose tolerance. Elevated rates of fat breakdown (lipolysis) lead to a release of free fatty acids (FFA's). These have a detrimental action on the uptake of insulin by the liver, which in turn results in increased glucogenesis (breakdown of amino acids and conversion to glucose), production of glucose by the liver, and systemic dyslipidaemia. These factors contribute to the prevailing systemic hyperinsulinemia (raised circulatory insulin concentrations) and decreased skeletal insulin sensitivity with reduced glucose uptake. Initially, the β-cells of the pancreas compensate for these processes by producing more insulin. In time, however, there is failure of the β-cells and the development of a raised circulating blood glucose concentration (hyperglycaemia), and hence NIDDM (Kopelman P. G. Nature. 404:639 (2000). Whereas non-obese individuals with NIDDM often only show secretory defect (β-cell failure), obese, NIDDM patients suffer from peripheral insulin resistance in combination with defective insulin secretion. Thus, NIDDM in obese and non-obese individuals may take two forms where the cause of hyperglycaemia differs: obesity-related NIDDM and non-obesity-related diabetes.

The invention features a method of diagnosing a subtype of NIDDM in an individual, i.e. grouping individuals into subtypes of diabetes based on the identity of markers in linkage disequilibrium with the insulin HphI locus and their body fat value. Such a method comprises genotyping at least one marker in linkage disquilibrium with the insulin HphI locus of the indvidual; determining a body fat value for the individual; and assigning a subtype based on the genotype, the body fat value and a predetermined value that correlates the genotype, the body fat value and likelihood of having a particular subtype of NIDDM.

In another aspect, the invention features a method of diagnosing a subtype of NIDDM in an individual as described above; however, the VNTR class of the insulin gene is determined and used to determine the subtype. For example, a subtype is based on the VNTR class, the body fat value and a predetermined value that correlates the VNTR class, the body fat value and likelihood of having a particular subtype of NIDDM, as described below.

TABLE IIA
Diagnosing a subtype of NIDDM

| | HAPLOTYPE | | |
|---|---|---|---|
| BMI (kg/m²) | [+/+] | [+/−] | [−/−] |
| 18.5 | NOR | NOR | NOR |
| 18.5-24.9 | NOR | NOR | NOR |
| 25.0-29.9 | OR | OR | OR |
| 30.0-39.9 | OR | OR | OR |
| >40.0 | OR | OR | OR |

OR indicates obesity-related NIDDM
NOR indicates non obesity-related NIDDM

TABLE IIB
Diagnosing a subtype of NIDDM

| | VNTR CLASS | | |
|---|---|---|---|
| BMI (kg/m²) | [I/I] | [I/III] | [III/III] |
| 18.5 | NOR | NOR | NOR |
| 18.5-24.9 | NOR | NOR | NOR |
| 25.0-29.9 | OR | OR | OR |
| 30.0-39.9 | OR | OR | OR |
| >40.0 | OR | OR | OR |

OR indicates obesity-related NIDDM
NOR indicates non obesity-related NIDDM

Treatment of Obesity-Related NIDDM

Obesity-related NIDDM cannot be cured, but the disease can be managed through efforts to reduce weight and maintain glucose homeostasis.

The proposed treatments for reducing body weight are of five types. (1) Food restriction is the most frequently used. The obese individuals are advised to change their dietary habits so as to consume fewer calories, i.e. a very low calorie (VLC) diet (400 and 800 kcal/day). Although this type of treatment is effective in the short-term, the recidivation rate is very high. (2) Increased calorie use through physical exercise is also proposed. This treatment is ineffective when applied alone, but it improves weight-loss in subjects on a low-calorie diet. Together, food restriction and increased calorie use are sometimes considered a single behavioral modification treatment. (3) Gastrointestinal surgery, which reduces the absorption of the calories ingested, is effective, but has been virtually abandoned because of the side effects it causes. (4) An approach that aims to reduce the absorption of dietary lipids by sequestering them in the lumen of the digestive tube is also in place. However, it induces physiological imbalances which are difficult to tolerate, including: deficiency in the absorption of fat-soluble vitamins, flatulence and steatorrhoea. Whatever the envisaged therapeutic approach, the treatments of obesity are all characterized by an extremely high recidivation rate. (5) There are five medicinal strategies that may lead to significant weight loss:

a) reducing food intake by amplifying inhibitory effects of anorexigenic signals or factors (those that suppress food intake) or by blocking orexigenic signals or factors (those that stimulate food intake), i.e. sibutramine;

b) blocking nutrient absorption (especially fat) in the gut, i.e. orlistat;

c) increasing thermogenesis by uncoupling of fuel metabolism from the generation of ATP, thereby dissipating food energy as heat, i.e. ephedrine and caffeine;

d) modulating fat or protein metabolism or storage by regulating fat synthesis/lipolysis or adipose differentiation/apoptosis; and

e) modulating the central controller regulating body weight by either altering the internal reference value sought by the controller or by modulating the primary afferent signals regarding fat stores that are analyzed by the controller (Bray G. A. et al., Nature. 404:672-674 (2000) and (Healtheon/WebMD. (1999)).

While physical exercise and reductions in dietary intake of calories will dramatically improve the diabetic condition, compliance with this treatment is very poor because of well-entrenched sedentary lifestyles and excess food consumption, especially high fat-containing food. Increasing the plasma level of insulin by administration of sulfonylureas (e.g. tolbutamide, glipizide) which stimulate the pancreatic β-cells to secrete more insulin or by injection of insulin aft r the response to sulfonylureas fails, will result in high enough insulin concentrations to stimulate the very insulin-resistant tissues. However, dangerously low levels of plasma glucose can result from these last two treatments and increasing insulin resistance due to the even higher plasma insulin levels could theoretically occur. The biguanides increase insulin sensitivity resulting in some correction of hyperglycemia. However, the two biguanides, phenformin and metformin, can induce lactic acidosis and nausea/diarrhea, respectively.

Methods for Determining a Body Fat Value

Obesity is loosely defined as an excess of fat over that needed to maintain health, while it is formally defined as a significant increase above ideal weight, ideal weight being defined as that which maximizes life expectancy (Friedman, J. M. Nature. 404:633 (2000). A convenient clinical and epidemiological measure of adiposity is the body mass index (BMI), which is calculated as weight divided by the square of the height (kg/m2). BMI is highly correlated with more complex measures of body fat, such as those described herein, although the relation is less accurate at the extremes of the height distribution. (Healtheon/WebMD 1999).

Body Mass Index

In clinical practice, body fat is most commonly and simply estimated by using a formula that combines weight and height. The underlying assumption is most variation in weight for persons of the same height is due to fat mass, and the formula most frequently used in studies is body-mass index (BMI). A graded classification of obesity using BMI values provides valuable information about increasing body fatness. It allows meaningful comparisons of weight status within and between populations and the identification of individuals and groups at risk of morbidity and mortality. It also permits identification of priorities for intervention at an individual or community level and for evaluating the effectiveness of such interventions. However, BMI may not correspond to the same degree of fatness across different populations. Nor does it account for the wide variation in the nature of obesity between different individuals and populations (Kopelman P. G. Nature. 404:635 (2000)).

The World Health Organization provides the following classifications of overweight using BMI:

TABLE A

| BMI (kg/m²) | W.H.O. classification | Popular description |
|---|---|---|
| 18.5 | Underweight | Thin |
| 18.5-24.9 | — | Healthy |
| 25.0-29.9 | Grade 1 overweight | Overweight |
| 30.0-39.9 | Grade 2 overweight | Obesity |
| >40.0 | Grade 3 overweight | Morbid obesity |

Other Methods of Measuring a Body Fat Value

In addition to BMI, there are number of methods of determining fat mass measurements including waist circumference, waist-to-hip ratio, skinfold thickness, and bioimpedance (Heymsfield S. B. et al. Am J Clin Nutr. 64:478-84 (1996)) and (Calle E. C. et al. New Engl J Med. 341:1097-1104(1999)and(Gallagher D. et al. Am J Epidemiol. 143:228-39(1996). Table B, herein, discusses each of these methods.

TABLE B

| Method | Definition | Advantages/limitations |
|---|---|---|
| BMI | Weight in kilograms divided by | BMI correlated strongly with |
| | square of the height in meters | densitometry measurements of fat |
| | | mass: main limitation is that it does |
| | | not distinguish fat mass from lean |
| | | mass |
| Waist | Measured (in centimeters) at midpoint | Waist circumference measures for |
| circumference | between lower border of ribs and | assessing upper body fat |
| | upper border of pelvis | deposition: neither provide precise |
| | | estimates of intra-abdominal |
| | | (visceral) fat |
| waist-to-hip ratio | Ratio of the waist circumference and | Waist-to-hip ratio is a good |
| | the hip circumference measured (in | indicator of abdominal (i.e., |
| | centimeters) at the upper border of | android, as opposed to gynecoid) |
| | pelvis | obesity, which is an even more |
| | | important risk factor for NIDDM |
| | | than obesity. |
| Skinfold | Measurement of skinfold thickness | Measurements are subject to |
| thickness | (in centimeters) with callipers | considerable variation between |
| | provides a more precise assessment if | observers, require accurate |
| | taken at multiple sites | callipers and do not provide any |
| | | information on abdominal and |
| | | intramuscular fat |
| Bioimpedance | Based on the principle that lean mass | Devices are simple and practical |
| | conducts current better than fat mass | but neither measure fat nor predict |
| | because it is primarily an electrolyte | biological outcomes more |
| | solution: measurement of resistance | accurately than simpler |
| | to a weak current (impedance) applied | anthropometric measurements |
| | across extremities provides an | |
| | estimate of body fat using an | |
| | empirically dervided equation | |

(Kopelman P. G. Nature. 404: 635 (2000))

Methods for Genotyping an Individual for Genetic Markers

Methods are provided to genotype a biological sample for one or more genetic markers of the present invention, all of which may be performed in vitro. Such methods of genotyping comprise determining the identity of a nucleotide at an insulin-related genetic marker site by any method known in the art. An insulin-related genetic marker is any marker in linkage disequilibrium with the insulin HphI locus. This includes any marker known in the art which is a surrogate for the VNTR in the insulin gene. A list of markers in linkage disquilibrium with the insulin HphI locus is provided in Table C, herein.

These methods find use in genotyping case-control populations in association studies as well as individuals in the context of detection of alleles of genetic markers which are known to be associated with a given trait, in which case both copies of the genetic marker present in an individual's genome are determined so that an individual may be classified as homozygous or heterozygous for a particular allele.

These genotyping methods can be performed on nucleic acid samples derived from a single individual or pooled DNA samples.

Genotyping can be performed using similar methods as those described above for the identification of the genetic markers, or using other genotyping methods such as those further described below. In preferred embodiments, the comparison of sequences of amplified genomic fragments from different individuals is used to identify new genetic markers whereas microsequencing is used for genotyping known genetic markers in diagnostic and association study applications.

Source of DNA for Genotyping

Any source of nucleic acids, in purified or non-purified form, can be utilized as the starting nucleic acid, provided it contains or is suspected of containing the specific nucleic acid sequence desired. DNA or RNA may be extracted from cells, tissues, body fluids and the like as described above. While nucleic acids for use in the genotyping methods of the invention can be derived from any mammalian source, the test subjects and individuals from which nucleic acid samples are taken are generally understood to be human.

Amplification of DNA Fragments Comprising Genetic Markers

Methods and polynucleotides are provided to amplify a segment of nucleotides comprising one or more genetic marker of the present invention. It will be appreciated that amplification of DNA fragments comprising genetic markers may be used in various methods and for various purposes and is not restricted to genotyping. Nevertheless, many genotyping methods, although not all, require the previous amplification of the DNA region carrying the genetic marker of interest. Such methods specifically increase the concentration or total number of sequences that span the genetic marker or include that site and sequences located either distal or proximal to it. Diagnostic assays may also rely on amplification of DNA segments carrying a genetic marker of the present invention. Amplification of DNA may be achieved by any method known in the art. Amplification techniques are described above in the section entitled, Amplification of the Insulin Gene.

Some of these amplification methods are particularly suited for the detection of single nucleotide polymorphisms and allow the simultaneous amplification of a target sequence and the identification of the polymorphic nucleotide as it is further described below.

The identification of genetic markers as described above allows the design of appropriate oligonucleotides, which can be used as primers to amplify DNA fragments comprising the genetic markers of the present invention. Amplification can be performed using the primers initially used to discover new genetic markers which are described herein or any set of primers allowing the amplification of a DNA fragment comprising a genetic marker of the present invention.

In some embodiments the present invention provides primers for amplifying a DNA fragment containing one or more genetic markers of the present invention. Preferred amplification primers are listed in Table C and Table Amplification Primers. It will be appreciated that the primers listed are merely exemplary and that any other set of primers which produce amplification products containing one or more genetic markers of the present invention.

The spacing of the primers determines the length of the segment to be amplified. In the context of the present invention, amplified segments carrying genetic markers can range in size from at least about 25 bp to 35 kbp. Amplification fragments from 25-3000 bp are typical, fragments from 50-1000 bp are preferred and fragments from 100-600 bp are highly preferred. It will be appreciated that amplification primers for the genetic markers may be any sequence which allow the specific amplification of any DNA fragment carrying the markers.

TABLE C

| Marker/ | | Annealing | PCR | | | |
|---|---|---|---|---|---|---|
| Position | Primers | Temp | product | Alleles | Enzyme | Method of detection |
| TH | TH1 | 60° C. | 106/110/114 | | | 6% acrylamide gel |
| microsatelite | TH2 | | 118/122 bp | | | |
| −9000 | | | | | | |
| −4217 | TH9B | 60° C. | 236 bp | T/C | PstI | 2% agarose gel in |
| PstI | TH10B | | | | (1 U) | 0.5X TBE |
| −2733 | INS68R | 60° C. | | A/C | | ARMS |
| | INS68C | | | | | |
| −2221 | INS56 | 63° C. | 186 bp | C/T | MspI | 2% agarose gel in |
| MspI | INS57 | | | | (1 U) | 0.5X TBE |
| −365 | | | | | | Southern blot |
| | | | | | | pINS310 |
| −23 | INS04 | 65° C. | 441 bp | | HphI | 2% agarose gel |
| HphI | INS05 | | | | (2.5 U) | The 9 bp band is not |
| | | | | | | detectable |
| +805 | | | | | DraIII | |
| DraIII | | | | | | |
| +1127 | | | | | PstI | |
| PstI | | | | | | |
| +1140 | INS71 | 60° C. | | A/C | | ARMS |
| | INS71RC | | | | | |
| +1355 | INS69 | 66° C. | | T/C | | ARMS |
| | INS69RC | | | | | |
| +1404 | | | | | | Fnu4H1 |
| +1428 | ins13 | 65.5° C. | 433 bp | | FokI | 1% agarose gel in |
| FoKI | DS02 | | | | (1 U) | 0.5X TBE |
| +2331 | INS73A | 60° C. | | A/T | | ARMS |
| | INS41 | | | | | |
| +2336 | INS55 | 64° C. | 116/121 bp | | | 4% agarose gel |
| | INS41 | | | | | (5 bp deletion) |
| +3201 | IIRI9 | 62° C. | | G/A | HaeII | |
| HaeII | IIRI2B | | | | | |
| +3580 | INS46 | 60° C. | | G/A | Msp1 | |
| Msp1 | INS47 | | | | | |
| +3688 | INS74C | 64° C. | | C/T | | ARMS |
| | INS74R | | | | | |
| +3839 | INS44 | 64° C. | | A/G | AlwN1 | |
| AlwN1 | INS45 | | | | | |
| +11000 | IGF2-26 | 64° C. | 91 bp | C/T | AluI | 3% agarose-1000 gel |
| AluI | IGF2-27 | | | | (1 U) | in 0.5X TBE |
| IGF2 exon 3 | | | | | | The 6 bp band is not |
| | | | | | | detectable |
| +32000 | ApalF | 55° C. | 236 bp | | ApaI | 2% agarose-1000 gel |
| ApaI | Apa1R | | | | (1 U) | in 0.5X TBE |

(Lucassen, A. M. et al. Nature Genet. 4, 305-310 (1993))

Methods of Genotyping DNA Samples for Genetic Markers

Any method known in the art can be used to identify the nucleotide present at a genetic marker site. Since the genetic marker allele to be detected has been identified and specified in the present invention, detection will prove simple for one of ordinary skill in the art by employing any of a number of techniques. Many genotyping methods require the previous amplification of the DNA region carrying the genetic marker of interest. While the amplification of target or signal is often preferred at present, ultrasensitive detection methods which do not require amplification or sequencing are also encompassed by the present genotyping methods. Methods well-known to those skilled in the art that can be used to detect genetic polymorphisms include methods such as, conventional dot blot analyzes, single strand conformational polymorphism analysis (SSCP) described by Orita et al. (1989) Proc. Natl. Acad. Sci U.S.A86: 2776-2770, denaturing gradient gel electrophoresis (DGGE), heteroduplex analysis, mismatch cleavage detection, and other conventional techniques as described in Sheffield, V. C. et al. (1991) Proc. Natl. Acad. Sci. U.S.A. 49:699-706, White, M. B. et al. (1992) Genomics. 12:301-306, Grompe, M. (1993) Nature Genetics. 5:111-117. Another method for determining the identity of the nucleotide present at a particular polymorphic site employs a specialized exonuclease-resistant nucleotide derivative as described in U.S. Pat. No. 4,656,127.

Preferred methods involve directly determining the identity of the nucleotide present at a genetic marker site by sequencing assay, allele-specific amplification assay, or hybridization assay. The following is a description of some preferred methods. A highly preferred method is the microsequencing technique. The term “sequencing” is used herein to refer to polymerase extension of duplex primer/template complexes and includes both traditional sequencing and microsequencing.

1) Sequencing Assays

The nucleotide present at a polymorphic site can be determined by sequencing methods. In a preferred embodiment, DNA samples are subjected to PCR amplification before sequencing as described above.

Preferably, the amplified DNA is subjected to automated dideoxy terminator sequencing reactions using a dye-primer cycle sequencing protocol. Sequence analysis allows the identification of the base present at the genetic marker site.

2) Microsequencing Assays

In microsequencing methods, the nucleotide at a polymorphic site in a target DNA is detected by a single nucleotide primer extension reaction. This method involves appropriate microsequencing primers which, hybridize just upstream of the polymorphic base of interest in the target nucleic acid. A polymerase is used to specifically extend the 3′ end of the primer with one single ddNTP (chain terminator) complementary to the nucleotide at the polymorphic site. Next the identity of the incorporated nucleotide is determined in any suitable way.

Typically, microsequencing reactions are carried out using fluorescent ddNTPs and the extended microsequencing primers are analyzed by electrophoresis on ABI 377 sequencing machines to determine the identity of the incorporated nucleotide as described in EP 412 883. Alternatively capillary electrophoresis can be used in order to process a higher number of assays simultaneously. An example of a typical microsequencing procedure that can be used in the context of the present invention is provided in Example 2.

Different approaches can be used for the labeling and detection of ddNTPs. A homogeneous phase detection method based on fluorescence resonance energy transfer has been described by Chen and Kwok (1997) Nucleic Acids Research.25:347-353 and Chen et al. (1997) Proc. Natl. Acad. Sci. USA.94(20): 10756-10761, the disclosures of which are incorporated herein by reference in their entireties. In this method, amplified genomic DNA fragments containing polymorphic sites are incubated with a 5′-fluorescein-labeled primer in the presence of allelic dye-labeled dideoxyribonucleoside triphosphates and a modified Taq polymerase. The dye-labeled primer is extended one base by the dye-terminator specific for the allele present on the template. At the end of the genotyping reaction, the fluorescence intensities of the two dyes in the reaction mixture are analyzed directly without separation or purification. All these steps can be performed in the same tube and the fluorescence changes can be monitored in real time. Alternatively, the extended primer may be analyzed by MALDI-TOF Mass Spectrometry. The base at the polymorphic site is identified by the mass added onto the microsequencing primer (see Haff L. A. and Smirnov I. P. (1997) Genome Research, 7:378-388), the disclosures of which are incorporated herein by reference in their entireties.

Microsequencing may be achieved by the established microsequencing method or by developments or derivatives thereof. Alternative methods include several solid-phase microsequencing techniques. The basic microsequencing protocol is the same as described previously, except that the method is conducted as a heterogenous phase assay, in which the primer or the target molecule is immobilized or captured onto a solid support. To simplify the primer separation and the terminal nucleotide addition analysis, oligonucleotides are attached to solid supports or are modified in such ways that permit affinity separation as well as polymerase extension. The 5′ ends and internal nucleotides of synthetic oligonucleotides can be modified in a number of different ways to permit different affinity separation approaches, e.g., biotinylation. If a single affinity group is used on the oligonucleotides, the oligonucleotides can be separated from the incorporated terminator regent. This eliminates the need of physical or size separation. More than one oligonucleotide can be separated from the terminator reagent and analyzed simultaneously if more than one affinity group is used. This permits the analysis of several nucleic acid species or more nucleic acid sequence information per extension reaction. The affinity group need not be on the priming oligonucleotide but could alternatively be present on the template. For example, immobilization can be carried out via an interaction between biotinylated DNA and streptavidin-coated microtitration wells or avidin-coated polystyrene particles. In the same manner oligonucleotides or templates may be attached to a solid support in a high-density format In such solid phase microsequencing reactions, incorporated ddNTs can be radiolabeled (Syvänen, Clinica Chimica Acta 226:225-236, 1994) or linked to fluorescein (Livak and Hainer, Human Mutation 3:379-385,1994). The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such as p-nitrophenyl phosphate). Other possible reporter-detection pairs include: ddNTP linked to dinitrophenyl (DNP) and anti-DNP alkaline phosphatase conjugate (Haiju et al., Clin. Chem. 39/11 2282-2287 (1993)) or biotinylated ddNTP and horseradish peroxidase-conjugated streptavidin with o-phenylenediamine as a substrate (WO 92/15712). As yet another alternative solid-phase microsequencing procedure, Nyren et al. (Analytical Biochemistry 208:171-175 (1993), described a method relying on the detection of DNA polymerase activity by an enzymatic luminometric inorganic pyrophosphate detection assay (ELIDA).

Pastinen et al. (Genome Research 7:606-614, 1997), describes a method for multiplex detection of single nucleotide polymorphism in which the solid phase minisequencing principle is applied to an oligonucleotide array format. High-density arrays of DNA probes attached to a solid support (DNA chips) are further below.

3) Allele-Specific Amplification Assay Methods

In one aspect the present invention provides polynucleotides and methods to determine the allele of one or more genetic markers of the present invention in a biological sample, by allele-specific amplification assays. Methods, primers and various parameters to amplify DNA fragments comprising genetic markers of the present invention are further described above in “Amplification of DNA Fragments Comprising Genetic Markers”.

Allele Specific Amplification Primers

Discrimination between the two alleles of a genetic marker can also be achieved by allele specific amplification, a selective strategy, whereby one of the alleles is amplified without amplification of the other allele. This is accomplished by placing the polymorphic base at the 3′ end of one of the amplification primers. Because the extension forms from the 3′ end of the primer, a mismatch at or near this position has an inhibitory effect on amplification. Therefore, under appropriate amplification conditions, these primers only direct amplification on their complementary allele. Determining the precise location of the mismatch and the corresponding assay conditions are well with the ordinary skill in the art.

Ligation/Amplification Based Methods

The “Oligonucleotide Ligation Assay” (OLA) uses two oligonucleotides which are designed to be capable of hybridizing to abutting sequences of a single strand of a target molecules. One of the oligonucleotides is biotinylated, and the other is detectably labeled. If the precise complementary sequence is found in a target molecule, the oligonucleotides will hybridize such that their termini abut, and create a ligation substrate that can be captured and detected. OLA is capable of detecting single nucleotide polymorphisms and may be advantageously combined with PCR as described by Nickerson D. A. et al. (1990) Proc. Natl. Acad. Sci. U.S.A. 87:8923-8927. In this method, PCR is used to achieve the exponential amplification of target DNA, which is then detected using OLA.

Other amplification methods which are particularly suited for the detection of single nucleotide polymorphism include LCR (ligase chain reaction), Gap LCR (GLCR) which are described above in “Amplification of the insulin gene”. LCR uses two pairs of probes to exponentially amplify a specific target The sequences of each pair of oligonucleotides, is selected to permit the pair to hybridize to abutting sequences of the same strand of the target. Such hybridization forms a substrate for a template-dependant ligase. In accordance with the present invention, LCR can be performed with oligonucleotides having the proximal and distal sequences of the same strand of a genetic marker site. In one embodiment, either oligonucleotide will be designed to include the genetic marker site. In such an embodiment, the reaction conditions are selected such that the oligonucleotides can be ligated together only if the target molecule either contains or lacks the specific nucleotide that is complementary to the genetic marker on the oligonucleotide. In an alternative embodiment, the oligonucleotides will not include the genetic marker, such that when they hybridize to the target molecule, a “gap” is created as described in WO 90/01069. This gap is then “filled” with complementary dNTPs (as mediated by DNA polymerase), or by an additional pair of oligonucleotides. Thus at the end of each cycle, each single strand has a complement capable of serving as a target during the next cycle and exponential allele-specific amplification of the desired sequence is obtained.

Ligase/Polymerase-mediated Genetic Bit Analysis™ is another method for determining the identity of a nucleotide at a preselected site in a nucleic acid molecule (WO 95/21271). This method involves the incorporation of a nucleoside triphosphate that is complementary to the nucleotide present at the preselected site onto the terminus of a primer molecule, and their subsequent ligation to a second oligonucleotide. The reaction is monitored by detecting a specific label attached to the reaction's solid phase or by detection in solution.

4) Hybridization Assay Methods

A preferred method of determining the identity of the nucleotide present at a genetic marker site involves nucleic acid hybridization. The hybridization probes, which can be conveniently used in such reactions, preferably include the probes defined herein. Any hybridization assay may be used including Southern hybridization, Northern hybridization, dot blot hybridization and solid-phase hybridization (see Sambrook, J., Fritsch, E. F., and T. Maniatis. (1989) Molecular Cloning. A Laboratory Manual. 2ed. Cold Spring Harbor Laboratory, Cold Spring Harbor, N.Y.).

Hybridization refers to the formation of a duplex structure by two single stranded nucleic acids due to complementary base pairing. Hybridization can occur between exactly complementary nucleic acid strands or between nucleic acid strands that contain minor regions of mismatch. Specific probes can be designed that hybridize to one form of a genetic marker and not to the other and therefore are able to discriminate between different allelic forms. Allele-specific probes are often used in pairs, one member of a pair showing perfect match to a target sequence containing the original allele and the other showing a perfect match to the target sequence containing the alternative allele. Hybridization conditions should be sufficiently stringent that there is a significant difference in hybridization intensity between alleles, and preferably an essentially binary response, whereby a probe hybridizes to only one of the alleles. Stringent, sequence specific hybridization conditions, under which a probe will hybridize only to the exactly complementary target sequence are well known in the art (Sambrook et al., 1989). Stringent conditions are sequence dependent and will be different in different circumstances. Generally, stringent conditions are selected to be about 5° C. lower than the thermal melting point (Tm) for the specific sequence at a defined ionic strength and pH. Although such hybridizations can be performed in solution, it is preferred to employ a solid-phase hybridization assay. The target DNA comprising a genetic marker of the present invention may be amplified prior to the hybridization reaction. The presence of a specific allele in the sample is determined by detecting the presence or the absence of stable hybrid duplexes formed between the probe and the target DNA. The detection of hybrid duplexes can be carried out by a number of methods. Various detection assay formats are well known which utilize detectable labels bound to either the target or the probe to enable detection of the hybrid duplexes. Typically, hybridization duplexes are separated from unhybridized nucleic acids and the labels bound to the duplexes are then detected. Those skilled in the art will recognize that wash steps may be employed to wash away excess target DNA or probe as well as unbound conjugate. Further, standard heterogeneous assay formats are suitable for detecting the hybrids using the labels present on the primers and probes.

Two recently developed assays allow hybridization-based allele discrimination with no need for separations or washes (see Landegren U. et al., Genome Research, 8:769-776,1998). The TaqMan assay takes advantage of the 5′ nuclease activity of Taq DNA polymerase to digest a DNA probe annealed specifically to the accumulating amplification product. TaqMan probes are labeled with a donor-acceptor dye pair that interacts via fluorescence energy transfer. Cleavage of the TaqMan probe by the advancing polymerase during amplification dissociates the donor dye from the quenching acceptor dye, greatly increasing the donor fluorescence. All reagents necessary to detect two allelic variants can be assembled at the beginning of the reaction and the results are monitored in real time (see Livak et al., Nature Genetics, 9:341-342, 1995). In an alternative homogeneous hybridization-based procedure, molecular beacons are used for allele discriminations. Molecular beacons are hairpin-shaped oligonucleotide probes that report the presence of specific nucleic acids in homogeneous solutions. When they bind to their targets they undergo a conformational reorganization that restores the fluorescence of an internally quenched fluorophore (Tyagi et al., Nature Biotechnology, 16:49-53, 1998).

The polynucleotides provided herein can be used in hybridization assays for the detection of genetic marker alleles in biological samples. These probes are characterized in that they preferably comprise between 8 and 50 nucleotides, and in that they are sufficiently complementary to a sequence comprising a genetic marker of the present invention to hybridize thereto and preferably sufficiently specific to be able to discriminate the targeted sequence for only one nucleotide variation. The GC content in the probes of the invention usually ranges between 10 and 75%, preferably between 35 and 60%, and more preferably between 40 and 55%. The length of these probes can range from 10, 15, 20, or 30 to at least 100 nucleotides, preferably from 10 to 50, more preferably from 18 to 35 nucleotides. A particularly preferred probe is 25 nucleotides in length. Preferably the genetic marker is within 4 nucleotides of the center of the polynucleotide probe. In particularly preferred probes the genetic marker is at the center of said polynucleotide. Shorter probes may lack specificity for a target nucleic acid sequence and generally require cooler temperatures to form sufficiently stable hybrid complexes with the template. Longer probes are expensive to produce and can sometimes self-hybridize to form hairpin structures. Methods for the synthesis of oligonucleotide probes have been described above and can be applied to the probes of the present invention.

By assaying the hybridization to an allele specific probe, one can detect the presence or absence of a genetic marker allele in a given sample. High-Throughput parallel hybridizations in array format are specifically encompassed within “hybridization assays” and are described below.

5) Hybridization to Addressable Arrays of Oligonucleotides

Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (e.g., the chip) at selected positions. Each DNA chip can contain thousands to millions of individual synthetic DNA probes arranged in a grid-like pattern and miniaturized to the size of a dime.

The chip technology has already been applied with success in numerous cases. For example, the screening of mutations has been undertaken in the BRCA1 gene, in S. cerevisiae mutant stains, and in the protease gene of HIV-1 virus (Hacia et al., Nature Genetics, 14(4):441-447, 1996; Shoemaker et al., Nature Genetics, 14(4):450-456, 1996 ; Kozal et al., Nature Medicine, 2:753-759, 1996). Chips of various formats for use in detecting genetic polymorphisms can be produced on a customized basis by Affymetrix (GeneChip™), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP 785280 describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of monomers, i.e. nucleotides. Tiling strategies are further described in PCT Publication No. WO 95/11995. In a particular aspect, arrays are tiled for a number of specific, identified genetic marker sequences. In particular, the array is tiled to include a number of detection blocks, each detection block being specific for a specific genetic marker or a set of genetic markers. For example, a detection block may be tiled to include a number of probes, which span the sequence segment that includes a specific polymorphism. To ensure probes that are complementary to each allele, the probes are synthesized in pairs differing at the genetic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the genetic marker. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the genetic marker are present in the sample. Hybridization and scanning may be carried out as described in PCT Publication No. WO 92/10092 and WO 95/11995 and U.S. Pat. No. 5,424,186.

Thus, in some embodiments, the chips may comprise an array of nucleic acid sequences of fragments of about 15 nucleotides in length. In further embodiments, the chip may comprise an array including at least one of the sequences selected from the group consisting of 9-27, 99-14387, 9-12, 9-13, 99-14405, and 9-16 and the sequences complementary thereto, or a fragment thereof, said fragment comprising at least about 8 consecutive nucleotides, preferably 10, 15, 20, more preferably 25, 30, 40, 47, or 50 consecutive nucleotides and containing a polymorphic base. In preferred embodiments the polymorphic base is within 5, 4, 3, 2, 1, nucleotides of the center of the said polynucleotide, more preferably at the center of said polynucleotide. In some embodiments, the chip may comprise an array of at least 2, 3, 4, 5, 6, 7, 8 or more of these polynucleotides of the invention.

6) Integrated Systems

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip to create functional microscopic valves and pumps with no moving parts. Varying the voltage controls the liquid flow at intersections between the micro-machined channels and changes the liquid flow rate for pumping across different sections of the microchip.

For genotyping genetic markers, the microfluidic system may integrate nucleic acid amplification, microsequencing, capillary electrophoresis and a detection method such as laser-induced fluorescence detection.

In a first step, the DNA samples are amplified, preferably by PCR. Then, the amplification products are subjected to automated microsequencing reactions using ddNTPs (specific fluorescence for each ddNTP) and the appropriate oligonucleotide microsequencing primers which hybridize just upstream of the targeted polymorphic base. Once the extension at the 3′ end is completed, the primers are separated from the unincorporated fluorescent ddNTPs by capillary electrophoresis. The separation medium used in capillary electrophoresis can for example be polyacrylamide, polyethyleneglycol or dextran. The incorporated ddNTPs in the single-nucleotide primer extension products are identified by fluorescence detection. This microchip can be used to process at least 96 to 384 samples in parallel. It can use the usual four color laser induced fluorescence detection of the ddNTPs.

Methods of Genetic Analysis Using the Genetic markers of the Present Invention

Different methods are available for the genetic analysis of complex traits (see Lander and Schork, Science, 265, 2037-2048, 1994). The search for disease-susceptibility genes is conducted using two main methods: the linkage approach in which evidence is sought for cosegregation between a locus and a putative trait locus using family studies, and the association approach in which evidence is sought for a statistically significant association between an allele and a trait or a trait causing allele (Khoury J. et al., Fundamentals of Genetic Epidemiology, Oxford University Press, N.Y., 1993). In general, the genetic markers of the present invention find use in any method known in the art to demonstrate a statistically significant correlation between a genotype and a phenotype. The genetic markers may be used in parametric and non-parametric linkage analysis methods. Preferably, the genetic markers of the present invention are used to identify genes associated with detectable traits using association studies, an approach which does not require the use of affected families and which permits the identification of genes associated with complex and sporadic traits.

The genetic analysis using the genetic markers of the present invention may be conducted on any scale. The whole set of genetic markers of the present invention or any subset of genetic markers of the present invention corresponding to the candidate gene may be used. Further, any set of genetic markers including a genetic marker of the present invention may be used. A set of genetic polymorphisms that could be used as genetic markers in combination with the genetic markers of the present invention has been described in WO 98/20165. As mentioned above, it should be noted that the genetic markers of the present invention may be included in any complete or partial genetic map of the human genome. These different uses are specifically contemplated in the present invention and claims.

The invention also comprises methods of detecting an association between a genotype and a phenotype, comprising the steps of a) genotyping at least one marker in linkage disquilibrium with the insulin HphI locus in a trait positive population according to a genotyping method of the invention; b) genotyping said marker in linkage disquilibrium with the insulin HphI locus in a control population according to a genotyping method of the invention; and c) determining whether a statistically significant association exists between the genotype and the phenotype. In addition, the methods of detecting an association between a genotype and a phenotype of the invention encompass methods with any further limitation described in this disclosure, or those following, specified alone or in any combination. Optionally, the marker in linkage disquilibrium with the insulin HphI locus may be selected from the markers provided in Table C; preferably markers −4217 PstI, −2221 MspI, −23 HphI, +1428 FokI, +11000 AluI and +32000 ApaI; or more preferably marker −23 HphI. Optionally, the marker in linkage disquilibrium with the insulin HphI locus may further include any other marker that is in linkage disquilibrium with the insulin HphI locus that is known in the art; as well as any marker determined to be in linkage disquilibrium with the insulin HphI locus by methods described herein. Optionally, the control population may be a trait negative population, or a random population. Optionally, each of the genotyping steps a) and b) may be performed on a pooled biological sample derived from each of the populations. Optionally, each of the genotyping of steps a) and by is performed separately on biological samples derived from each individual in the population or a subsample thereof.

The invention also encompasses methods of estimating the frequency of a haplotype for a set of genetic markers in a population, comprising the steps of: a) genotyping at least two markers in linkage disquilibrium with the insulin HphI locus for each individual in the population or a subsample thereof, according to a genotyping method of the invention; and b) applying a haplotype determination method to the identities of the nucleotides determined in steps a) to obtain an estimate of the frequency. In addition, the methods of estimating the frequency of a haplotype of the invention encompass methods with any further limitation described in this disclosure, or those following, specified alone or in any combination: Optionally, the marker in linkage disquilibrium with the insulin HphI locus may be selected from the markers provided in Table C; preferably markers −4217 PstI, −2221 MspI, −23 HphI, +1428 FokI, +11000 AluI and +32000 ApaI; or more preferably marker −23 HphI. Optionally, the marker in linkage disquilibrium with the insulin HphI locus may further include any other marker that is in linkage disquilibrium with the insulin HphI locus that is known in the art; as well as any marker determined to be in linkage disquilibrium with the insulin HphI locus by methods described herein. Optionally, the haplotype determination method is performed by asymmetric PCR amplification, double PCR amplification of specific alleles, the Clark algorithm, or an expectation-maximization algorithm.

An additional embodiment of the present invention encompasses methods of detecting an association between a haplotype and a phenotype, comprising the steps of: a) estimating the frequency of at least one haplotype in a trait positive population, according to a method of the invention for estimating the frequency of a haplotype; b) estimating the frequency of the haplotype in a control population, according to a method of the invention for estimating the frequency of a haplotype; and c) determining whether a statistically significant association exists between the haplotype and the phenotype. In addition, the methods of detecting an association between a haplotype and a phenotype of the invention encompass methods with any further limitation described in this disclosure, or those following. Optionally, the genetic marker may be selected from the markers provided in Table C; preferably markers −4217 PstI, −2221 MspI, −23 HphI, +1428 FokI, +11000 AluI and +32000 ApaI; or more preferably marker −23 HphI. Optionally, the marker in linkage disquilibrium with the insulin HphI locus may further include any other marker that is in linkage disquilibrium with the insulin HphI locus that is known in the art; as well as any marker determined to be in linkage disquilibrium with the insulin HphI locus by methods described herein. Optionally, the control population is a trait negative population, or a random population. Optionally, the phenotype is an insulin-related disorder. Optionally, the method comprises the additional steps of determining the phenotype in the trait positive and the control populations prior to step c). Optionally, wherein the insulin-related disorder is hyperinsulinemia.

Linkage Analysis

Linkage analysis is based upon establishing a correlation between the transmission of genetic markers and that of a specific trait throughout generations within a family. Thus, the aim of linkage analysis is to detect marker loci that show cosegregation with a trait of interest in pedigrees.

Parametric Methods

When data are available from successive generations there is the opportunity to study the degree of linkage between pairs of loci. Estimates of the recombination fraction enable loci to be ordered and placed onto a genetic map. With loci that are genetic markers, a genetic map can be established, and then the strength of linkage between markers and traits can be calculated and used to indicate the relative positions of markers and genes affecting those (Weir, B. S., Genetic data Analysis II: Methods for Discrete population genetic Data, Sinauer Assoc., Inc., Sunderland, Mass., U.S.A., 1996). The classical method for linkage analysis is the logarithm of odds (lod) score method (see Morton N. E., Am. J Hum. Genet., 7:277-318, 1955; Ott J., Analysis of Human Genetic Linkage, John Hopkins University Press, Baltimore, 1991). Calculation of lod scores requires specification of the mode of inheritance for the disease (parametric method). Generally, the length of the candidate region identified using linkage analysis is between 2 and 20 Mb. Once a candidate region is identified as described above, analysis of recombinant individuals using additional markers allows further delineation of the candidate region. Linkage analysis studies have generally relied on the use of a maximum of 5,000 microsatellite markers, thus limiting the maximum theoretical attainable resolution of linkage analysis to about 600 kb on average.

Linkage analysis has been successfully applied to map simple genetic traits that show clear Mendelian inheritance patterns and which have a high penetrance (i.e., the ratio between the number of trait positive carriers of allele a and the total number of a carriers in the population). However, parametric linkage analysis suffers from a variety of drawbacks. First, it is limited by its reliance on the choice of a genetic model suitable for each studied trait. Furthermore, as already mentioned, the resolution attainable using linkage analysis is limited, and complementary studies are required to refine the analysis of the typical 2 Mb to 20 Mb regions initially identified through linkage analysis. In addition, parametric linkage analysis approaches have proven difficult when applied to complex genetic traits, such as those due to the combined action of multiple genes and/or environmental factors. It is very difficult to model these factors adequately in a lod score analysis. In such cases, too large an effort and cost are needed to recruit the adequate number of affected families required for applying linkage analysis to these situations, as recently discussed by Risch, N. and Merikangas, K. (Science, 273:1516-1517, 1996).

Non-Parametric Methods

The advantage of the so-called non-parametric methods for linkage analysis is that they do not require specification of the mode of inheritance for the disease, they tend to be more useful for the analysis of complex traits. In non-parametric methods, one tries to prove that the inheritance pattern of a chromosomal region is not consistent with random Mendelian segregation by showing that affected relatives inherit identical copies of the region more often than expected by chance. Affected relatives should show excess “allele sharing” even in the presence of incomplete penetrance and polygenic inheritance. In non-parametric linkage analysis the degree of agreement at a marker locus in two individuals can be measured either by the number of alleles identical by state (IBS) or by the number of alleles identical by descent (IBD). Affected sib pair analysis is a well-known special case and is the simplest form of these methods.

The genetic markers of the present invention may be used in both parametric and non-parametric linkage analysis. Preferably genetic markers may be used in non-parametric methods which allow the mapping of genes involved in complex traits. The genetic markers of the present invention may be used in both IBD- and IBS- methods to map genes affecting a complex trait. In such studies, taking advantage of the high density of genetic markers, several adjacent genetic marker loci may be pooled to achieve the efficiency attained by multi-allelic markers (Zhao et al., Am. J Hum. Genet., 63:225-240, (1998).

Population Association Studies

The present invention comprises methods for identifying if the insulin gene or a particular allelic variant thereof is associated with a detectable trait using the genetic markers of the present invention. In one embodiment the present invention comprises methods to detect an association between a genetic marker allele or a genetic marker haplotype and a trait Further, the invention comprises methods to identify a trait causing allele in linkage disequilibrium with any genetic marker allele of the present invention.

As described above, alternative approaches can be employed to perform association studies: genome-wide association studies, candidate region association studies and candidate gene association studies. In a preferred embodiment, the genetic markers of the present invention are used to perform candidate gene association studies. The candidate gene analysis clearly provides a short-cut approach to the identification of genes and gene polymorphisms related to a particular trait when some information concerning the biology of the trait is available. Further, the genetic markers of the present invention may be incorporated in any map of genetic markers of the human genome in order to perform genome-wide association studies. Methods to generate a high-density map of genetic markers has been described in PCT Publication No. WO 00/28080. The genetic markers of the present invention may further be incorporated in any map of a specific candidate region of the genome (a specific chromosome or a specific chromosomal segment for example).

As mentioned above, association studies may be conducted within the general population and are not limited to studies performed on related individuals in affected families. Association studies are extremely valuable as they permit the analysis of sporadic or multifactor traits. Moreover, association studies represent a powerful method for fine-scale mapping enabling much finer mapping of trait causing alleles than linkage studies. Studies based on pedigrees often only narrow the location of the trait causing allele. Association studies using the genetic markers of the present invention can therefore be used to refine the location of a trait causing allele in a candidate region identified by Linkage Analysis methods. Moreover, once a chromosome segment of interest has been identified, the presence of a candidate gene, such as a candidate gene of the present invention, in the region of interest can provide a shortcut to the identification of the trait causing allele. Genetic markers of the present invention can be used to demonstrate that a candidate gene is associated with a trait. Such uses are specifically contemplated in the present invention.

Determining the Frequency of a Genetic Marker Allele or of a Genetic Marker Haplotype in a Population

Association studies explore the relationships among frequencies for sets of alleles between loci.

Determining the Frequency of an Allele in a Population

Allelic frequencies of the genetic markers in a populations can be determined using one of the methods described above under the heading “Methods for Genotyping an Individual for Genetic Markers,” or any genotyping procedure suitable for this intended purpose. Genotyping pooled samples or individual samples can determine the frequency of a genetic marker allele in a population. One way to reduce the number of genotypings required is to use pooled samples. A major obstacle in using pooled samples is in terms of accuracy and reproducibility for determining accurate DNA concentrations in setting up the pools. Genotyping individual samples provides higher sensitivity, reproducibility and accuracy and; is the preferred method used in the present invention. Preferably, each individual is genotyped separately and simple gene counting is applied to determine the frequency of an allele of a genetic marker or of a genotype in a given population.

Determining the Frequency of a Haplotype in a Population

The gametic phase of haplotypes is unknown when diploid individuals are heterozygous at more than one locus. Using genealogical information in families gametic phase can sometimes be inferred (Perlin et al., Am. J Hum Genet., 55:777-787, 1994). When no genealogical information is available different strategies may be used. One possibility is that the multiple-site heterozygous diploids can be eliminated from the analysis, keeping only the homozygotes and the single-site. heterozygote individuals, but this approach might lead to a possible bias in the sample composition and the underestimation of low-frequency haplotypes. Another possibility is that single chromosomes can be studied independently, for example, by asymmetric PCR amplification (see Newton et al., Nucleic Acids Res., 17:2503-2516, 1989; Wu et al., Proc. Natl. Acad. Sci. USA, 86:2757, 1989), or by isolation of single chromosome by limit dilution followed by PCR amplification (see Ruano et al., Proc. Natl. Acad. Sci. USA, 87:6296-6300, 1990). Further, a sample may be haplotyped for sufficiently close genetic markers by double PCR amplification of specific alleles (Sarkar, G. and Sommer S. S., Biotechniques, 1991). These approaches are not entirely satisfying either because of their technical complexity, the additional cost they entail, their lack of generalisation at a large scale, or the possible biases they introduce. To overcome these difficulties, an algorithm to infer the phase of PCR-amplified DNA genotypes introduced by Clark A. G. (Mol. Biol. Evol., 7:111-122, 1990), may be used. Briefly, the principle is to start filling a preliminary list of haplotypes present in the sample by examining unambiguous individuals, that is, the complete homozygotes and the single-site heterozygotes. Then other individuals in the same sample are screened for the possible occurrence of previously recognised haplotypes. For each positive identification, the complementary haplotype is added to the list of recognised haplotypes, until the phase information for all individuals is either resolved or identified as unresolved. This method assigns a single haplotype to each multiheterozygous individual, whereas several haplotypes are possible when there are more than one heterozygous site. Alternatively, one can use methods estimating haplotype frequencies in a population without assigning haplotypes to each individual. Preferably, a method based on an expectation-maximization (EM) algorithm (Dempster et al., J. R. Stat. Soc., 39B: 1-38, 1977), leading to maximum-likelihood estimates of haplotype frequencies under the assumption of Hardy-Weinberg proportions (random mating) is used (see Excoffier L. and Slatkin M., Mol. Biol. Evol., 12(5): 921-927, 1995). The EM algorithm is a generalised iterative maximum-likelihood approach to estimation that is useful when data are ambiguous and/or incomplete. The EM algorithm is used to resolve heterozygotes into haplotypes. Haplotype estimations are further described below under the heading “Statistical methods.” Any other method known in the art to determine or to estimate the frequency of a haplotype in a population may also be used.

Linkage Disequilibrium Analysis

Linkage disequilibrium is the non-random association of alleles at two or more loci and represents a powerful tool for mapping genes involved in disease traits (see Ajioka R. S. et al., Am. J. Hum. Genet., 60:1439-1447, 1997). Genetic markers, because they are densely spaced in the human genome and can be genotyped in greater numbers than other types of genetic markers (such as RFLP or VNTR markers), are particularly useful in genetic analysis based on linkage disequilibrium

When a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombination events occur between the disease mutation and these marker polymorphisms, and the disequilibrium gradually dissipates. The pace of this dissipation is a function of the recombination frequency, so the markers closest to the disease gene will manifest higher levels of disequilibrium than those that are further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine-scale mapping of a disease locus, it is useful to have some knowledge of the patterns of linkage disequilibrium that exist between markers in the studied region. As mentioned above the mapping resolution achieved through the analysis of linkage disequilibrium is much higher than that of linkage studies. The high density of genetic markers combined with linkage disequilibrium analysis provides powerful tools for fine-scale mapping. Different methods to calculate linkage disequilibrium are described below under the heading “Statistical Methods.”

Population-Based Case-Control Studies of Trait-Marker Associations

As mentioned above, the occurrence of pairs of specific alleles at different loci on the same chromosome is not random and the deviation from random is called linkage disequilibrium. Association studies focus on population frequencies and rely on the phenomenon of linkage disequilibrium. If a specific allele in a given gene is directly involved in causing a particular trait, its frequency will be statistically increased in an affected (trait positive) population, when compared to the frequency in a trait negative population or in a random control population. As a consequence of the existence of linkage disequilibrium, the frequency of all other alleles present in the haplotype carrying the trait-causing allele will also be increased in trait positive individuals compared to trait negative individuals or random controls. Therefore, association between the trait and any allele (specifically a genetic marker allele) in linkage disequilibrium with the trait-causing allele will suffice to suggest the presence of a trait-related gene in that particular region. Case-control populations can be genotyped for genetic markers to identify associations that narrowly locate a trait causing allele. As any marker in linkage disequilibrium with one given marker associated with a trait will be associated with the trait. Linkage disequilibrium allows the relative frequencies in case-control populations of a limited number of genetic polymorphisms (specifically genetic markers) to be analyzed as an alternative to screening all possible functional polymorphisms in order to find trait-causing alleles. Association studies compare the frequency of marker alleles in unrelated case-control populations, and represent powerful tools for the dissection of complex traits.

Case-Control Populations (Inclusion Criteria)

Population-based association studies do not concern familial inheritance but compare the prevalence of a particular genetic marker, or a set of markers, in case-control populations. They are case-control studies based on comparison of unrelated case (affected or trait positive) individuals and unrelated control (unaffected, trait negative or random) individuals. Preferably the control group is composed of unaffected or trait negative individuals. Further, the control group is ethnically matched to the case population. Moreover, the control group is preferably matched to the case-population for the main known confusion factor for the trait under study (for example age-matched for an age-dependent trait). Ideally, individuals in the two samples are paired in such a way that they are expected to differ only in their disease status. The terms “trait positive population,” “case population” and “affected population” are used interchangeably herein.

An important step in the dissection of complex traits using association studies is the choice of case-control populations (see Lander and Schork, Science, 265, 2037-2048, 1994). A major step in the choice of case-control populations is the clinical definition of a given trait or phenotype. Any genetic trait may be analyzed by the association method proposed here by carefully selecting the individuals to be included in the trait positive and trait negative phenotypic groups. Four criteria are often useful: clinical phenotype, age at onset, family history and severity. The selection procedure for continuous or quantitative traits (such as blood pressure for example) involves selecting individuals at opposite ends of the phenotype distribution of the trait under study, so as to include in these trait positive and trait negative populations individuals with non-overlapping phenotypes. Preferably, case-control populations consist of phenotypically homogeneous populations. Trait positive and trait negative populations consist of phenotypically uniform populations of individuals representing each between 1 and 98%, preferably between 1 and 80%, more preferably between 1 and 50%, and more preferably between 1 and 30%, most preferably between 1 and 20% of the total population under study, and preferably selected among individuals exhibiting non-overlapping phenotypes. The clearer the difference between the two trait phenotypes, the greater the probability of detecting an association with genetic markers. The selection of those drastically different but relatively uniform phenotypes enables efficient comparisons in association studies and the possible detection of marked differences at the genetic level, provided that the sample sizes of the populations under study are significant enough.

In preferred embodiments, a first group of between 50 and 300 trait positive individuals, preferably about 100 individuals, are recruited according to their phenotypes. A similar number of trait negative individuals are included in such studies.

In the present invention, typical examples of inclusion criteria include obesity, diabetic, ethnicity, monotonic gain of weight, age, gender and puberty.

Suitable examples of association studies using genetic markers including the genetic markers of the present invention, are studies involving the following populations:

a case population suffering from juvenile onset obesity and a lean control population, or

a case population suffering from juvenile obesity and an insulin-related disorder and a control population suffering from juvenile obesity but is not suffering from an insulin-related disorder, or

a case population suffering from obesity-related NIDDM and a non-diabetic control population.

In an embodiment, markers in linkage disequilibrium with the insulin HphI locus may be used to identify individuals who are prone to insulin-related disorders. This includes diagnostic and prognostic assays to identify individuals who possess factors which predispose them to alterations of insulin secretion in response to fat accumulation, as well as clinical trials and treatment regimes which utilize these assays. Drug treatment may include any pharmaceutical compound suspected or known in the art used to treat obesity or control insulin-related disorders.

Association Analysis

The general strategy to perform association studies using genetic markers derived from a region carrying a candidate gene is to scan two groups of individuals (case-control populations) in order to measure and statistically compare the allele frequencies of the genetic markers of the present invention in both groups.

If a statistically significant association with a trait is identified for at least one or more of the analyzed genetic markers, one can assume that: either the associated allele is directly responsible for causing the trait (i.e. the associated allele is the trait causing allele), or more likely the associated allele is in linkage disequilibrium with the trait causing allele. The specific characteristics of the associated allele with respect to the candidate gene function usually give further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker, and performing further association studies with the polymorphisms that are revealed in an iterative manner.

Association studies are usually run in two successive steps. In a first phase, the frequencies of a reduced number of genetic markers from the candidate gene are determined in the trait positive and trait negative populations. In a second phase of the analysis, the position of the genetic loci responsible for the given trait is further refined using a higher density of markers from the relevant region.

Based on Example 3, herein, subgroups for clinical studies or association studies could be identified based on either the identity of at least one marker in linkage disequilibrium with the insulin HphI locus or the VNTR class of the insulin gene and the subject's body fat value. Specifically, subjects who are HphI [+/+] homozygotes (insulin VNTR I/I) show a stronger correlation between insulin and BMI than those with HphI [+/−] or [−/−] genotypes (insulin VNTR I/III and insulin VNTR III/III, respectively) and a comparable adiposity. Therefore, obese individuals with HphI [+/−] or [−/−] genotypes are significantly more likely to develop NIDDM than obese individuals with HphI [+/+] genotypes and may be selected for inclusion in clinical studies or association studies accordingly.

The invention features a method of selecting an individual for inclusion in a clinical study or an association study that involves an insulin-related disorder, comprising: a) determining the identity of the polymorphic base(s) of at least one marker in linkage disquilibrium with the insulin HphI locus of the individual; b) determining a body fat value for the individual; and c) including the individual in the study based on the identity of the polymorphic bases, the body fat value and a predetermined value that correlates the identity, the body fat value and the risk of developing an insulin-related disorder. In another aspect, th invention features a method of selecting an individual for inclusion in a clinical study or an association study that involves an insulin-related disorder, comprising: a) determining the VNTR class of an insulin gene of the individual; b) determining a body fat value for the individual; and c) including the individual in the study based on the VNTR class, the body fat value and a predetermined value that correlates the VNTR class, the body fat value and the risk of developing an insulin-related disorder. See Table IA for a predetermined value that correlates identity of the polymorphic base(s) of at least one marker in linkage disquilibrium with the insulin HphI locus, the body fat value and the risk of developing an insulin-related disorder. See Table IB for a predetermined value that correlates the VNTR class, the body fat value and the risk of developing an insulin-related disorder.

Haplotype Analysis

As described above, when a chromosome carrying a disease allele fist appears in a population as a result of either mutation or migration, the mutant allele necessarily resides on a chromosome having a set of linked markers: the ancestral haplotype. This haplotype can be tracked through populations and its statistical association with a given trait can be analyzed. Complementing single point (allelic) association studies with multi-point association studies also called haplotype studies increases the statistical power of association studies. Thus, a haplotype association study allows one to define the frequency and the type of the ancestral carrier haplotype. A haplotype analysis is important in that it increases the statistical power of an analysis involving individual markers.

In a first stage of a haplotype frequency analysis, the frequency of the possible haplotypes based on various combinations of the identified genetic markers of the invention is determined. The haplotype frequency is then compared for distinct populations of trait positive and control individuals. The number of trait positive individuals, which should be, subjected to this analysis to obtain statistically significant results usually ranges between 30 and 300, with a preferred number of individuals ranging between 50 and 150. The same considerations apply to the number of unaffected individuals (or random control) used in the study. The results of this first analysis provide haplotype frequencies in case-control populations, for each evaluated haplotype frequency a p-value and an odd ratio are calculated. If a statistically significant association is found the relative risk for an individual carrying the given haplotype of being affected with the trait under study can be approximated.

Interaction Analysis

The genetic markers of the present invention may also be used to identify patterns of genetic markers associated with detectable traits resulting-from polygenic interactions. The analysis of genetic interaction between alleles at unlinked loci requires individual genotyping using the techniques described herein. The analysis of allelic interaction among a selected set of genetic markers with appropriate level of statistical significance can be considered as a haplotype analysis. Interaction analysis consists in stratifying the case-control populations with respect to a given haplotype for the first loci and performing a haplotype analysis with the second loci with each subpopulation.

Testing For Linkage in the Presence of Association

The genetic markers of the present invention may further be used in TDT (transmission/disequilibrium test). TDT tests for both linkage and association and is not affected by population stratification. TDT requires data for affected individuals and their parents or data from unaffected sibs instead of from parents (see Spielmann S. et al., 1993; Schaid D. J. et al., 1996, Spielmann S. and Ewens W. J., 1998). Such combined tests generally reduce the false-positive errors produced by separate analyses.

Statistical Methods

In general, any method known in the art to test whether a trait and a genotype show a statistically significant correlation may be used.

1) Methods In Linkage Analysis

Statistical methods and computer programs useful for linkage analysis are well-known to those skilled in the art (see Terwilliger J. D. and Ott J., Handbook of Human Genetic Linkage, John Hopkins University Press, London, 1994; Ott J., Analysis of Human Genetic Linkage, John Hopkins University Press, Baltimore, 1991).

2) Methods to Estimate Haplotype Frequencies in a Population

As described above, when genotypes are scored, it is often not possible to distinguish heterozygotes so that haplotype frequencies cannot be easily inferred. When the gametic phase is not known, haplotype frequencies can be estimated from the multilocus genotypic data. Any method known to person skilled in the art can be used to estimate haplotype frequencies (see Lange K, Mathematical and Statistical Methods for Genetic Analysis, Springer, N.Y., 1997; Weir, B. S., Genetic data Analysis II: Methods for Discrete population genetic Data, Sinauer Assoc., Inc., Sunderland, Mass., U.S.A, 1996). Preferably, maximum-likelihood haplotype frequencies are computed using an Expectation- Maximization (EM) algorithm (see Dempster et al., J. R. Stat. Soc., 39B:1-38, 1977; Excoffier L. and Slatkin M., Mol. Biol. Evol., 12(5): 921-927, 1995). This procedure is an iterative process aiming at obtaining maximum-likelihood estimates of haplotype frequencies from multi-locus genotype data when the gametic phase is unknown. Haplotype estimations are usually performed by applying the EM algorithm using for example the EM-HAPLO program (Hawley M. E. et al., Am. J. Phys. Anthropol., 18:104, 1994) or the Arlequin program (Schneider et al., Arlequin: a software for population genetics data analysis, University of Geneva, 1997). The EM algorithm is a gen ralised iterative maximum likelihood approach to estimation and is briefly described below.

In what follows, phenotypes will refer to multi-locus genotypes with unknown haplotypic phase. Genotypes will refer to mutli-locus genotypes with known haplotypic phase.

Suppose one has a sample of N unrelated individuals typed for K markers. The data observed are the unknown-phase K-locus phenotypes that can be categorized with F different phenotypes. Further, suppose that we have H possible haplotypes (in the case of K genetic markers, we have for the maximum number of possible haplotypes H=2^{K}).

For phenotype j with cⱼ possible genotypes, we have:

$${P}_{j} = {\sum }_{i = 1}^{{c}_{j}} P ( genotype ( i ) ) = {\sum }_{i = 1}^{{c}_{j}} P ( {h}_{k} , {h}_{l} ) . \qquad Equation 1$$

Here, Pⱼ is the probability of the j^{th} phenotype, and P(hₖhₗ) is the probability of the i^{th} genotype composed of haplotypes hₖ and hₗ. Under random mating (i.e. Hardy-Weinberg Equilibrium), P(hₖhₗ) is expressed as:

P(hₖ,hₗ)=P(hₖ)² for hₖ=hₗ, and P(hₖ,hₗ)=2P(hₖ)P(hₗ) for hₖ≠hₗ. Equation 2

The E-M algorithm is composed of the following steps: First, the genotype frequencies are estimated from a set of initial values of haplotype frequencies. These haplotype frequencies are denoted P₁⁽⁰⁾, P₂⁽⁰⁾, P₃⁽⁰⁾, . . . , P_{H}⁽⁰⁾. The initial values for the haplotype frequencies may be obtained from a random number generator or in some other way well known in the art. This step is referred to the Expectation step. The next step in the method, called the Maximization step, consists of using the estimates for the genotype frequencies to re-calculate the haplotype frequencies. The first iteration haplotype frequency estimates are denoted by P₁⁽¹⁾, P₂^{(l), P}₃^{(l)}, . . . , P_{H}⁽¹⁾. In general, the Expectation step at the s^{th} iteration consists of calculating the probability of placing each phenotype into the different possible genotypes based on the haplotype frequencies of the previous iteration:

$${P ( {h}_{k} , {h}_{l} )}^{( s )} = \frac{{n}_{j}}{N} [ \frac{{{P}_{j} ( {h}_{k} , {h}_{l} )}^{( s )}}{{P}_{j}} ] , \qquad Equation 3$$

where nⱼ is the number of individuals with the j^{th} phenotype and Pⱼ(hₖ,hₗ)^{(s)} is the probability of genotype hₖhₗ in phenotype j. In the Maximization step, which is equivalent to the gene-counting method (Smith, Ann. Hum. Genet., 21:254-276, 1957), the haplotype frequencies are re-estimated based on the genotype estimates:

$${P}_{t}^{( s + 1 )} = \frac{1}{2} {\sum }_{j = 1}^{F} {\sum }_{i = 1}^{{c}_{j}} {δ}_{i t} {{P}_{j} ( {h}_{k} , {h}_{l} )}^{( s )} . \qquad Equation 4$$

Here, δᵢₜ is an indicator variable which counts the number of occurrences that haplotype t is present in i^{th} genotype; it takes on values 0, 1, and 2.

The E-M iterations cease when the following criterion has been reached. Using Maximum Likelihood Estimation (MLE) theory, one assumes that the phenotypes j are distributed multinomially. At each iteration s, one can compute the likelihood function L. Convergence is achieved when the difference of the log-likehood between two consecutive iterations is less than some small number, preferably 10⁻⁷.

3) Methods To Calculate Linkage Disequilibrium Between Markers

A number of methods can be used to calculate linkage disequilibrium between any two genetic positions, in practice linkage disequilibrium is measured by applying a statistical association test to haplotype data taken from a population.

Linkage disequilibrium between any pair of genetic markers comprising at least one of the genetic markers of the present invention (Mᵢ, Mⱼ) having alleles (aᵢ/bᵢ) at marker Mᵢ and alleles (aⱼ/bⱼ) at marker Mⱼ can be calculated for every allele combination (aᵢ,aⱼ; aᵢ,bⱼ; bᵢ,aⱼ and bᵢ,bⱼ), according to the Piazza formula:

Δₐᵢₐⱼ=√θ4−√(θ4+θ3) (θ4+θ2), where:

θ4=−−=frequency of genotypes not having allele aᵢ at Mᵢ and not having allele aⱼ at Mⱼ

θ3=−+=frequency of genotypes not having allele aᵢ at Mᵢ and having allele aⱼ at Mⱼ

θ2=+−=frequency of genotypes having allele aᵢ at Mᵢ and not having allele aⱼ at Mⱼ

Linkage disequilibrium (LD) between pairs of genetic markers (Mᵢ, Mⱼ) can also be calculated for every allele combination (ai,aj; ai,bj; bi,aj and bi,bj), according to the maximum-likelihood estimate (MLE) for delta (the composite genotypic disequilibrium coefficient), as described by Weir (Weir B. S., 1996). The MLE for the composite linkage disequilibrium is:

Dₐᵢₐⱼ=(2nₗ+n₂+n₃+n₄/2)/N−2(pr(aᵢ). pr(aⱼ))

Where n₁=Σ phenotype (aᵢ/aᵢ, aⱼ/aⱼ), n₂=Σ phenotype (aᵢ/aⱼ, aⱼ/bⱼ), n₃=Σ phenotype (aᵢ/bᵢ, aⱼ/aⱼ), n4=Σ phenotype (aᵢ/bᵢ, aⱼ/bⱼ) and N is the number of individuals in the sample.

This formula allows linkage disequilibrium between alleles to be estimated when only genotype, and not haplotype, data are available.

Another means of calculating the linkage disequilibrium between markers is as follows. For a couple of genetic markers, Mᵢ(aᵢ/bᵢ) and Mⱼ (aⱼ/bⱼ), fitting the Hardy-Weinberg equilibrium, one can estimate the four possible haplotype frequencies in a given population according to the approach described above.

The estimation of gametic disequilibrium between ai and aj is simply:

Dₐᵢₐⱼ=pr(haplotype(aᵢ,aⱼ))−pr(aᵢ).pr(aⱼ).

Where pr(aᵢ) is the probability of allele aᵢ and pr(aⱼ) is the probability of allele aⱼ and where pr(haplotype (aᵢ, aⱼ)) is estimated as in Equation 3 above.

For a couple of genetic markers only one measure of disequilibrium is necessary to describe the association between Mₗ and Mⱼ. Then a normalized value of the above is calculated as follows:

D′ₐᵢₐⱼ=Dₐᵢₐⱼ/max (−pr(aᵢ). pr(aⱼ), −pr(bᵢ). pr(bⱼ)) with Dₐᵢₐⱼ<0

D′ₐᵢₐⱼ=Dₐᵢₐⱼ/max (pr(bᵢ). pr(aⱼ), pr(aᵢ). pr(bⱼ)) with Dₐᵢₐⱼ>0

The skilled person will readily appreciate that other LD calculation methods can be used.

Linkage disequilibrium among a set of genetic markers having an adequate heterozygosity rate can be determined by genotyping between 50 and 1000 unrelated individuals, preferably between 75 and 200, more preferably around 100.

4) Testing For Association

Methods for determining the statistical significance of a correlation between a phenotype and a genotype, in this case an allele at a genetic marker or a haplotype made up of such alleles, may be determined by any statistical test known in the art and with any accepted threshold of statistical significance being required. The application of particular methods and thresholds of significance are well with in the skill of the ordinary practitioner of the art.

Testing for association is performed by determining the frequency of a genetic marker allele in case and control populations and comparing these frequencies with a statistical test to determine if their is a statistically significant difference in frequency which would indicate a correlation between the trait and the genetic marker allele under study. Similarly, a haplotype analysis is performed by estimating the frequencies of all possible haplotypes for a given set of genetic markers in case and control populations, and comparing these frequencies with a statistical test to determine if their is a statistically significant correlation between the haplotype and the phenotype (trait) under study. Any statistical tool useful to test for a statistically significant association between a genotype and a phenotype may be used. Preferably the statistical test employed is a chi-square test with one degree of freedom. A P-value is calculated (the P-value is the probability that a statistic as large or larger than the observed one would occur by chance).

Statistical Significance

In preferred embodiments, significance for diagnosis purposes, either as a positive basis for further diagnostic tests or as a preliminary starting point for early preventive therapy, the p value related to a genetic marker association is preferably about 1×10⁻² or less, more preferably about 1×10⁻⁴ or less, for a single genetic marker analysis and about 1×10⁻³ or less, still more preferably 1×10⁻⁶ or less and most preferably of about 1×10⁻⁸ or less, for a haplotype analysis involving two or more markers. These values are believed to be applicable to any association studies involving single or multiple marker combinations.

The skilled person can use the range of values set forth above as a starting point in order to carry out association studies with genetic markers of the present invention. In doing so, significant associations between the genetic markers of the present invention and obesity or disorders related to obesity can be revealed and used for diagnosis and drug screening purposes.

Phenotypic Permutation

In order to confirm the statistical significance of the first stage haplotype analysis described above, it might be suitable to perform further analyses in which genotyping data from case-control individuals are pooled and randomized with respect to the trait phenotype. Each individual genotyping data is randomly allocated to two groups, which contain the same number of individuals as the case-control populations used to compile the data obtained in the first stage. A second stage haplotype analysis is preferably run on these artificial groups, preferably for the markers included in the haplotype of the first stage analysis showing the highest relative risk coefficient This experiment is reiterated preferably at least between 100 and 10000 times. The repeated iterations allow the determination of the percentage of obtained haplotypes with a significant p-value level below about 1×10⁻³.

Assessment of Statistical Association

To address the problem of false positives similar analysis may be performed with the same case-control populations in random genomic regions. Results in random regions and the candidate region are compared as described in PCT Publication No. WO 00/28080.

5) Evaluation of Risk Factors

The association between a risk factor (in genetic epidemiology the risk factor is the presence or the absence of a certain allele or haplotype at marker loci) and a disease is measured by the odds ratio (OR) and by the relative risk (RR). If P(R⁺) is the probability of developing the disease for individuals with R and P(R⁻) is the probability for individuals without the risk factor, then the relative risk is simply the ratio of the two probabilities, that is:

RR=P(R⁺)/P(R⁻)

In case-control studies, direct measures of the relative risk cannot be obtained because of the sampling design. However, the odds ratio allows a good approximation of the relative risk for low-incidence diseases and can be calculated:

OR=(F⁺/(1−F⁺))/(F⁻/(1−F))

F⁺ is the frequency of the exposure to the risk factor in cases and F⁻ is the frequency of the exposure to the risk factor in controls. F⁺ and F⁻ are calculated using the allelic or haplotype frequencies of the study and further depend on the underlying genetic model (dominant, recessive, additive, etc).

One can further estimate the attributable risk (AR) which describes the proportion of individuals in a population exhibiting a trait due to a given risk factor. This measure is important in quantifying the role of a specific factor in disease etiology and in terms of the public health impact of a risk factor. The public health relevance of this measure lies in estimating the proportion of cases of disease in the population that could be prevented if the exposure of interest were absent. AR is determined as follows:

AR=P_{E}(RR−1)/ (P_{E}(RR−1)+1)

AR is the risk attributable to a genetic marker allele or a genetic marker haplotype. P_{E} is the frequency of exposure to an allele or a haplotype within the population at large; and RR is the relative risk which, is approximated with the odds ratio when the trait under study has a relatively low incidence in the general population.

Identification of Genetic Markers in Linkage Disequilibrium with the Genetic Markers of the Invention

Once a first genetic marker has been identified in a genomic region of interest, a practitioner of ordinary skill in the art, using the teachings of the present invention, can easily identify additional genetic markers in linkage disequilibrium with this first marker. As mentioned before any marker in linkage disequilibrium with a first marker associated with a trait will be associated with the trait. Therefore, once an association has been demonstrated between a given genetic marker and a trait, the discovery of additional genetic markers associated with this trait is of great interest in order to increase the density of genetic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first genetic marker from a plurality of individuals; (b) identifying of second genetic markers in the genomic region harboring the first genetic marker; (c) conducting a linkage disequilibrium analysis between the first genetic marker and second genetic markers; and (d) selecting the second genetic markers as being in linkage disequilibrium with the first marker. Subcombinations comprising steps (b) and (c) are also contemplated.

Methods to identify genetic markers and to conduct linkage disequilibrium analysis are described herein and can be carried out by the skilled person without undue experimentation. The present invention also concerns genetic markers which are in linkage disequilibrium with the insulin HphI locus, which are expected to present similar characteristics in terms of their respective association with a given trait. The HphI locus is in strong linkage disequilibrium with the neighboring insulin VNTR the ‘+’ alleles (T) of the HphI locus are in complete linkage disequilibrium with class I allels of the neighboring insulin VNTR, and ‘−’ alleles (A) with the class III alleles. Therefore, linkage disequilibrium analysis also tests the insulin VNTR through the −23 HphI polymorphism as a surrogate marker. Optionally, wherein the marker in linkage disquilibrium with the insulin HphI locus is selected from the group consisting of markers described in Table C; preferably markers −4217 PstI, −2221 MspI, −23 HphI, +1428 FokI, +11000 AluI and +32000 ApaI; or more preferably marker −23 HphI. Optionally, the marker in linkage disquilibrium with the insulin HphI locus may further include any other marker that is in linkage disquilibrium with the insulin HphI locus that is known in the art; as well as any marker determined to be in linkage disquilibrium with the insulin HphI locus by methods described herein.

Mapping Studies: Identification of Functional Mutations

Once a positive association is confirmed with a genetic marker of the present invention, sequence in the associated candidate region (within linkage disequillibrium of the insulin gene) can be scanned for mutations by comparing the sequences of a selected number of trait positive and trait negative individuals. In a preferred embodiment, functional regions such as exons and splice sites, promoters and other regulatory regions of the insulin gene are scanned for mutations. Preferably, trait positive individuals carry the haplotype shown to be associated with the trait, and trait negative individuals do not carry the haplotype or allele associated with the trait The mutation detection procedure is essentially similar to that used for biallelic site identification.

The method used to detect such mutations generally comprises the following steps: (a) amplification of a region of the candidate gene comprising a genetic marker or a group of genetic markers associated with the trait from DNA samples of trait positive patients and trait negative controls; (b) sequencing of the amplified region; (c) comparison of DNA sequences from trait-positive patients and trait-negative controls; and (d) determination of mutations specific to trait-positive patients. Subcombinations which comprise steps (b) and (c) are specifically contemplated.

It is preferred that candidate polymorphisms be then verified by screening a larger population of cases and controls by means of any genotyping procedure such as those described herein, preferably using a microsequencing technique in an individual test format. Polymorphisms are considered as candidate mutations when present in cases and controls at frequencies compatible with the expected association results.

Genetic Markers of the Invention in Methods of Genetic Diagnostics

The genetic markers of the present invention can also be used to develop diagnostic tests capable of identifying individuals who express a detectable trait as the result of a specific genotype or individuals whose genotype places them at risk of developing a detectable trait at a subsequent time.

It will of course be understood by practitioners skilled in the treatment or diagnosis of obesity and disorders related to obesity that the present invention does not intend to provide an absolute identification of individuals who could be at risk of developing a particular disease involving obesity and disorders related to obesity but rather to indicate a certain degree or likelihood of developing a disease. However, this information is extremely valuable as it can, in certain circumstances, be used to initiate preventive treatments or to allow an individual carrying a significant haplotype to foresee warning signs such as minor symptoms. In diseases in which attacks may be extremely severe and sometimes fatal if not treated on time, the knowledge of a potential predisposition, even if this predisposition is not absolute, might contribute in a very significant manner to treatment efficacy.

The diagnostic techniques of the present invention may employ a variety of methodologies to determine whether a test subject has a genetic marker pattern associated with an increased risk of developing a detectable trait or whether the individual suffers from a detectable trait as a result of a particular mutation, including methods which enable the analysis of individual chromosomes for haplotyping, such as family studies, single sperm DNA analysis or somatic hybrids. The trait analyzed using the present diagnostics may be any detectable trait, including obesity and disorders related to obesity.

Another aspect of the present invention relates to a method of determining whether an individual is at risk of developing a trait or whether an individual expresses a trait as a consequence of possessing a particular trait-causing allele. The present invention also relates to a method of determining whether an individual is at risk of developing a plurality of traits or whether an individual expresses a plurality of traits as a result of possessing a particular trait-causing allele. These methods involve obtaining a nucleic acid sample from the individual and determining whether the nucleic acid sample contains one or more alleles of one or more genetic markers indicative of a risk of developing the trait or indicative that the individual expresses the trait as a result of possessing a particular trait-causing allele. These methods also involve obtaining a nucleic acid sample from the individual and, determining, whether the nucleic acid sample contains at least one allele or at least one genetic marker haplotype, indicative of a risk of developing the trait or indicative that the individual expresses the trait as a result of possessing a particular insulin polymorphism or mutation (trait-causing allele).

Preferably, in such diagnostic methods, a nucleic acid sample is obtained from the individual and this sample is genotyped using methods described above in “Methods Of Genotyping DNA Samples For Genetic Markers.” The diagnostics may be based on a single genetic marker or on a group of genetic markers. In each of these methods, a nucleic acid sample is obtained from the test subject and the genetic marker pattern of one or more of the markers in linkage disquilibrium with the insulin HphI locus is determined. Alternatively, the one or more genetic markers are selected from the group of markers described in Table C; preferably markers −4217 PstI, −2221 MspI, −23 HphI, +1428 FokI, +11000 AluI and +32000 ApaI; or more preferably marker −23 HphI. Optionally, the marker in linkage disquilibrium with the insulin HphI locus may further include any other marker that is in linkage disquilibrium with the insulin HphI locus that is known in the art; as well as any marker determined to be in linkage disquilibrium with the insulin HphI locus by methods described herein.

In one embodiment, a PCR amplification is conducted on the nucleic acid sample to amplify regions in which polymorphisms associated with a detectable phenotype have been identified. The amplification products are sequenced to determine whether the individual possesses one or more insulin polymorphisms associated with a detectable phenotype. The primers used to generate amplification products may comprise the primers listed in Table C and Table Amplification Primers. Alternatively, the nucleic acid sample is subjected to microsequencing reactions as described above to determine whether the individual possesses one or more insulin polymorphisms associated with a detectable phenotype resulting from a mutation or a polymorphism in the insulin gene.

In another embodiment, the nucleic acid sample is contacted with one or more allele specific oligonucleotide probes which specifically hybridize to one or more insulin alleles associated with a detectable phenotype. In another embodiment, the nucleic acid sample is contacted with a second insulin oligonucleotide capable of producing an amplification product when used with the allele specific oligonucleotide in an amplification reaction. The presence of an amplification product in the amplification reaction indicates that the individual possesses one or more insulin-related alleles associated with a detectable phenotype.

As described herein, the diagnostics may be based on a single genetic marker or a group of genetic markers. Preferably, the genetic marker or combination of gentic markers is selected from the group consisting of markers in linkage disquilibrium with the insulin HphI locus described in Table C; preferably markers −4217 PstI, −2221 MspI, −23 HphI, +1428 FokI, +11000 AluI and +32000 ApaI; or more preferably marker −23 HphI. Optionally, the marker in linkage disquilibrium with the insulin HphI locus may further include any other marker that is in linkage disquilibrium with the insulin HphI locus that is known in the art; as well as any marker determined to be in linkage disquilibrium with the insulin HphI locus by methods described herein. Diagnostic kits may comprise any of the polynucleotides of the present invention.

These diagnostic methods are extremely valuable as they can, in certain circumstances, be used to initiate preventive treatments or to allow an individual carrying a significant genotype or haplotype to foresee warning signs such as minor symptoms. For example, in the study described in Example 3, the subjects were all obese juveniles who had not yet developed NIDDM. However, by identifying the obese juveniles who are at risk for insulin-related disorders, particularly obesity-related NIDDM, they could be targeted now for more intensive treatment to prevent the onset of later severe disease.

Diagnostics, which analyze and predict response to a drug or side effects to a drug, may be used to determine whether an individual should be treated with a particular drug. For example, if the diagnostic indicates a likelihood that an individual will respond positively to treatment with a particular drug, the drug may be administered to the individual. Conversely, if the diagnostic indicates that an individual is likely to respond negatively to treatment with a particular drug, an alternative course of treatment may be prescribed. A negative response may be defined as either the absence of an efficacious response or the presence of toxic side effects. For example, in the study described in Example 3, the identified markers in linkage disquilibrium with the insulin HphI locus would be useful for genotyping a population of obese people to determine which people are more likely to be susceptibile to drugs designed to manage insulin-related disorders. Other associations between markers in linkage disquilibrium with the insulin HphI locus and other traits associated with insulin-related disorders can also be determined using the methods of the invention without undue experimentation and would indicate other markers useful to identify sub-populations of people likely to be susceptible (or not) to a drug targeting those traits. In addition, specific associations can be performed looking at drug outcome (treatment/side effect) to identify other useful markers for predicting risks/successful treatment.

Clinical drug trials represent another application for the markers of the present invention. One or more markers indicative of response to an agent acting against an insulin-related disorder or to side effects to an agent acting against an insulin-related disorder may be identified using the methods described above. Thereafter, potential participants in clinical trials of such an agent may be screened to identify those individuals most likely to respond favorably to the drug and/or exclude those likely to experience side effects. In that way, the effectiveness of drug treatment may be measured in individuals who have the potential to respond positively to the drug, without lowering the measurement as a result of the inclusion of individuals who are unlikely to respond positively in the study and/or without risking undesirable safety problems.

### EXAMPLES

### Example 1

### De Novo Identification of Genetic Markers

The genetic markers set forth in this application were isolated from human genomic sequences. To identify genetic markers, genomic fragments were amplified, sequenced and compared in a plurality of individuals.

Sequencing

PCR products were obtained using primers listed in Table Amplification Primers. Amplification across sequences obtained from GENBANK and across previously unsequenced regions of DNA using primers in flanking known sequences allowed determination of the sequence of a contiguous stretch of chromosome spanning 12.5 kb. Primers were designed such that they either included a 5′ non-template stretch of nucleotides forming a unique restriction site or so that they flanked such a site in the sequence. To obtain sequence between IGF2 exon I and the Alu repeat, a region covering 5.3 kb, amplification of a clone covering this region (λINS-2) was performed using gp-32 as described previously. This fragment was digested, subcloned into M13 and sequenced, such that PCR primers could be designed for amplification of smaller segments of genomic DNA from patients. PCR products were digested with appropriate enzymes, gel purified and cloned into M13 mp 18 and 19. They were sequenced using dideoxy chain termination technique (Sequenase, USB). Sequences were compared in 8 clones from each of 4 individuals. A base pair change in only one of 8 clones was assumed to be due to Taq polymerase infidelity. If a difference was observed in 2 or more clones it was assumed to be a potential polymorphism and further investigated.

Table Amplification Primers

| | Primer | |
|---|---|---|
| Region Amplified | Used for Amplification | Annealing Temp. Ta (° C.) |
| −4476 to −3194 | THX13/THX14A | 68 |
| −3304 to −2231 | THX14B/TH09 | 67 |
| −2285 to −1737 | TH03/TH04 | 65 |
| −1789 to −1186 | TH07/TH08 | 65 |
| −748 to +1460 | | |
| +1460 to +2232 | IGFP1A/AL04 | 68 |
| +2181 to +2715 | RT03/RT02 | 70 |
| +2690 to +3314 | IGFALU/PR18 | 68 |
| +3313 to +3872 | PR17/PR15 | 65 |
| +3779 to +4912 | PR16/BamAL8 | 67 |
| +4770 to +5552 | INTP4/INTDrev | 68 |
| +5460 to +6127 | PR25/INTPR3 | 68 |
| +6074 to +6568 | INTP3rev/INTArev | 65 |
| +6488 to +7309 | INTPR1/ALUINS | 65 |
| +7281 to +8024 | ALU5′/ALU3′ | 70 |

### Example 2

### Genotyping of Genetic Markers

New polymorphisms were screened to determine whether they altered a restriction enzyme site in the sequence. These sites were then amplified in a panel of random diabetics and controls and a subset of polymorphisms were amplified in families, using primers listed in Table Genotyping Primers. Typical PCR conditions: 96-well microtiter plates (Perkin), each 50ul reaction containing 200 ng DNA, 1.5 mM MgCl2, 5 ul 10× reaction Buffer (Perkin Elmer), 10% DMSO (Pst1), 02 mM each dNTP, 1 uM of each primer and 1.25 U of Taq Polymerase (Perkin Elmer). 30-35 cycles were performed using a 9700 Perkin Elmer thermocycler. 10 ul of PCR products were digested with 1-2.5 U of appropriate enzymes and gel electrophoresed to determine genotype. Where a restriction site was not affected, allele specific amplification was performed. 5′ VNTR genotyping was performed by Southern blotting and hybridization with probe pINS310, and −4217/Pst1 genotyping by Southern blotting and hybridization with probe pJ2.4 or by PCR.

TABLE

Genotyping Primers

| | | | | | |
|---|---|---|---|---|---|
| | | Annealing | PCR | | Method of |
| Position | Primers (5′-3′) | Temp | product | Enzyme | detection |

| | | | | | |
|---|---|---|---|---|---|
| −4217 | TH9B: TGACGCCAAGGACAAGCTCA | 60° C. | 236 bp | Pstl | 2% agarose |
| Pstl | (SEQ ID NO:1) | | | (1 U) | gel 0.5X |
| | TH10B: CCAGCAGCCCCAGTCCTGCA | | | | TBE |
| | (SEQ ID NO:2) | | | | |
| −2221 | INS56: CACCAGCTGGCCTTCAAGGT | 63° C. | 186 bp Mspl | 2% agarose | |
| MspI | (SEQ ID NO:3) | | | (1 U) | gel in 0.5X |
| | INS57: GCTGGGCACTAACAAGGTGT | | | | TBE |
| | (SEQ ID NO:4) | | | | |
| −23 | INS04: TCCAGGACAGGCTGCATCAG | 65° C. | 441 bp | Hphl | 2% agarose |
| Hphl | (SEQ ID NO:5) | | | (2.5 U) | gel in 0.5X |
| | INS05: AGCAATGGGCGGTTGGCTCA | | | | TBE |
| | (SEQ ID NO:6) | | | | The 9 bp band |
| | | | | | is not |
| | | | | | detectable |
| +1428 | INS13: TAAAGCCCTTGAACCAGC | 65.5° C. | 433 bp | Fok1 | 1% agarose |
| Foki | (SEQ ID NO:7) | | | (1 U) | gel in 0.5X |
| | DS02: CAGCCCAGcCTCCTCCCTCCACA | | | | TBE |
| | (SEQ ID NO:8) | | | | |
| +11000 | IGF2-26: CCCAGOGGCCGAAGAGTCA | 64° C. | 64° C. | Alul | 3% agarose- |
| Alul | (SEQ ID NO:9) | | | (1 U) | 1000 gel in |
| | IGF2-27: GCTGAGCTGGCAGCGATTCA | | | | 0.5X TBE |
| | (SEQ ID NO:10) | | | | The 6 bp band |
| | | | | | is not |
| | | | | | detectable |
| +32000 | Apa1F: CTTGGACTTTGAGTCAAATTGG | 55° C. | 236 bp | Apal | 2% agarose- |
| Apal | (SEQ ID NO: 11) | | | (1 U) | 1000 gel in |
| | Apa1R: CCTCCTTTGGTCTTACTGGG | | | | 0.5X TBE |
| | (SEQ ID NO: 12) | | | | |

The [+] alleles indicate the restriction enzyme cuts the sequence, whereas [−] alleles indicate a cut was not made. The resulting band length at each position is provided below.

Table Allele Frequency

| Position | Products of digestion |
|---|---|
| −4217 | +/+: 152 and 84 bp |
| Pstl | +/−: 236, 152, and 84 bp |
| | −/−: 236 bp |
| −2221 | +/+: 108 and 78 bp |
| MspI | +/−: 186, 108, and 78 bp |
| | −/−: 186 bp |
| −23 | +/+: 232, 161, 39, and 9 bp |
| HphI | +/−: 271, 232, 161, 39, and 9 bp |
| | −/−: 271, 161, and 9 bp |
| +1428 | +/+: 266, and 167 bp |
| FokI | +/−: 433, 266, and 167 bp |
| | −/−: 433 bp |
| +11000 | +/+: 58, 27, and 6 bp |
| AluI | +/−: 85, 58, 27, and 6 bp |
| | −/−: 85 and 6 bp |
| +32000 | +/+: 171 and 65 bp |
| ApaI | +/−: 236, 171, and 65 bp |
| | −/−: 236 bp |

### Example 3

### Association Study Between the Insulin Gene VNTR and Fasting Insulin Levels in Obese Juveniles

Subjects and Methods

Two caucasian cohorts of obese patients were recruited based on analysis of patronymic names and family history: one (n=201) from Mediterranean families (Italy, Spain, Portugal, Algeria) and one from Central Europe origin (France, Belgium, Germany, Poland) (n=257). These two cohorts had comparable multi-site insulin gene region haplotypes (determined from the study of 6 neighbouring SNPs by haplotype estimation and likehood ratio testing of equality between haplotype profile, not shown), reflecting their close genetic origin. Because of similarities of insulin to body mass index (BMI) relationships in the two cohorts, they were pooled into a single analysis as Genob Cohort I. These 458 caucasian children had body weight >85th percentile before 6 yrs, demonstrated a monotonic gain of weight and were never subjected to weight reduction attempts. Glucose and insulin were measured in fasting conditions. Patients were genotyped at the −23 HphI locus as described in Example 2, herein. In caucasians, the ‘+’ alleles ( of the HphI locus are in complete linkage disequilibrium with class I alleles of the neighbouring insulin VNTR, and ‘−’ alleles with the class III alleles: only 0.23% insulin region haplotypes are discordant between HphI ‘+’ and VNTR class I alleles. Therefore, this study tests the insulin VNTR through the −23 HphI polymorphism as a surrogate marker.

Results

In young obese individuals, HphI allele and genotype frequencies were comparable to those in 568 lean Caucasian subjects (Table Genotype Frequency); thus suggesting this polymorphism and the neighbouring VNTR are not related to common forms of juvenile obesity. It does not exclude this possibility, however, if other factors are taken into account, or in other populations.

Table Genotype Frequency

| Genotype | Weight = obese | | |
|---|---|---|---|
| at HphI | (BMI > | Weight = normal | Weight = lean |
| locus | 25 kg/m2) | (BMI = 13-25 kg/m2) | (BMI < 20 kg/m2) |
| [+/+] | 53% | 49% | 53% |
| [+/−] | 39% | 43% | 35% |
| [−/−] | 8% | 8% | 12% |

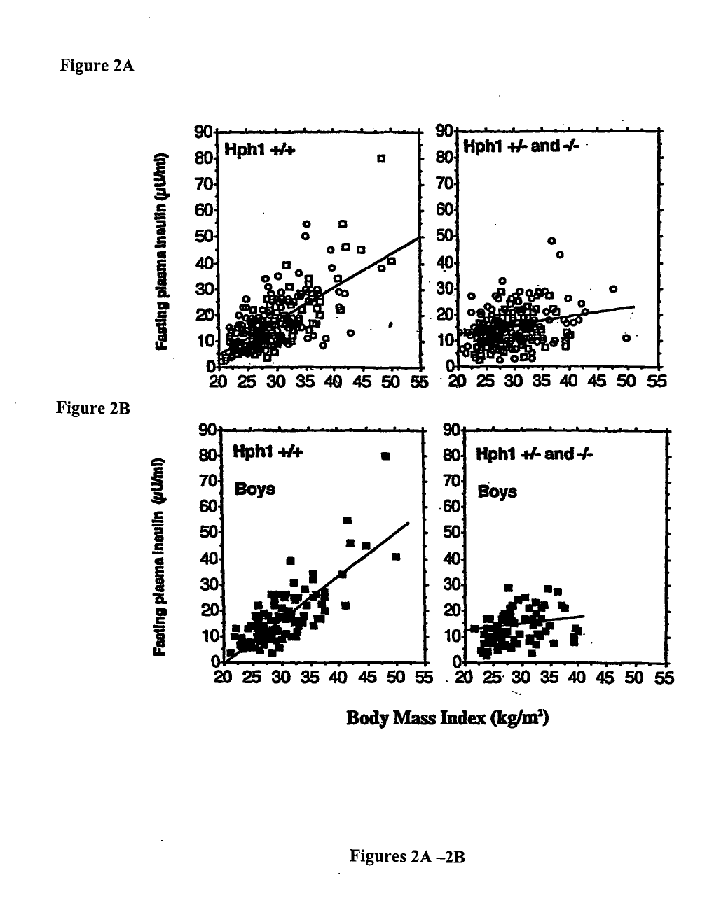

HphI genotypes were associated with differences in fasting insulin levels (Table 1): patients with HphI [+/+] genotypes, although younger, showed higher insulin levels than those with HphI[+/−] or [−/−] genotypes and a comparable adiposity (Table 1). The difference was more pronounced in superobese children whose fasting insulin levels were approximatively 60-70% higher in HphI [+/+] individuals (Table 1). In the whole obese cohort, plasma insulin and BMI were correlated (r=0.54, p<0.0001) as expected. Covariance analysis showed that HphI genotype had a major influence on the relationship between insulin and BMI (p<0.0001). HphI [+/+] homozygotes (insulin VNTR I/I) showed a stronger correlation between insulin and BMI than the two other genotypes (FIG. 2A).

TABLE 1
Main characteristics of the obese children in the two cohorts.

| | Cohort I | | | Cohort II | | |
|---|---|---|---|---|---|---|
| HphI genotype | +/+ | +/− | −/− | +/+ | +/− | −/− |
| n | 238 | 186 | 34 | 79 | 62 | 16 |
| Age (years) | 11.6 ± 0.2 | 12.3 ± 0.2 | 11.9 ± 0.5 | 11.9 ± 0.3 | 12.0 ± 0.4 | 12.2 ± 0.7 |
| BMI (kg/m²) | 29.6 ± 0.4 | 29.7 ± 0.4 | 30.0 ± 0.9 | 31.1 ± 0.6 | 29.9 ± 0.6 | 30.7 ± 1.1 |
| Fasting insulin | 17.0 ± 1.0 | 15.0 ± 1.0 | 15.0 ± 1.0 | 19.9 ± 1.3 | 15.6 ± 0.7 | 14.5 ± 1.4 |
| (μU/ml) | | | | | | |

The difference between genotypic groups was tested by recasting the situation as a general linear model to measure the influence of additional factors, such as age, sex, and puberty, on the regression of insulin on BMI (Table 2). The significance of the influence of HphI genotypes on the BMI-insulin relationship under this model is reflected in the interaction term of BMI*HphI as it contributes to the prediction of fasting insulin (p<0.0001). A highly significant (p<0.001) association of the insulin level relation to BMI was also observed with the neighbouring markers (−4217 PstI, −2221 MspI, +1428 FokI, +11000 AluI) that all are in strong LD with HphI alleles. A slightly weaker association (p=0.03) was observed with +32000 Apal polymorphism which has a smaller degree of LD with −23 HphI.

TABLE 2
Main characteristics of the superobese children
(BMI ≧ 96^{th} percentile) in the two cohorts

| | |
|---|---|
| Cohort I | Cohort II |

| HphI genotype | +/+ | +/− | −/− | +/+ | +/− | −/− |
|---|---|---|---|---|---|---|
| n | 53 | 44 | 8 | 23 | 17 | 4 |
| Age (years) | 13.8 ± 0.2 | 15.0 ± 0.4 | 14.1 ± 0.6 | 13.8 ± 0.4 | 14.7 ± 0.5 | 14.9 ± 1.6 |
| BMI (kg/m²) | 37.6 ± 0.6 | 36.7 ± 0.6 | 37.3 ± 1.6 | 37.5 ± 0.9 | 36.5 ± 0.7 | 36.4 ± 2.2 |
| Fasting | 28.2 ± 2.0 | 17.0 ± 1.0 | 19.0 ± 3.0 | 28.5 ± 3.0 | 16.6 ± 1.5 | 15.7 ± 2.0 |
| insulin | | | | | | |
| (μU/ml) | | | | | | |

In addition to insulin genotypes, gender had a strong effect on the relationship beween insulin and BMI. Obese boys showed a stronger correlation (n=192, r=0.65 p<0.0001) than girls (n=266, r=0.47, p<0.0001) as well as steeper regression slopes (FIG. 2B). The difference was significant (p<0.0002) for the BMI*sex interactive term 0in the model of Table 1B. The possibility of a combined effect for genotype and gender on the BMI-insulin relationship was addressed via the regression analysis model as well. Although there do appear to be differences in correlation strength for the four gender and genotype subgroups, they reflect differences in the two main factors separately, rather than interactively (Table 3). In other words, the relative effect of genotype on the BMI-insulin relationship does not differ between boys and girls.

Because of the importance of a replication cohort in association studies, an additional group of 157 young obese patients (GenOb Cohort II) was recruited from a multicentric French program. These patients were of mixed Caucasian origin, either from European (n=127) or Algerian (n=30) families. They were studied using similar inclusion criteria and techniques as Cohort I. The main characteristics of Cohort II patients are shown in Table 1 and 2. The distribution of genotype prevalence was comparable and the results confirmatory of those observed in the initial cohort. More specifically, the influence of the HphI genotype on the relationship between fasting insulin and BMI was significant (Table 3) and fitted regression parameters comparable with those reported in Cohort I (see the “Figure Legends” for comparison of values). BMI explained approximatively 53% of the variance of plasma insulin in HphI [+/+] obese children (48% in Cohort I), versus only 2% in the patients with patients with [+/−] or [−/−]genotypes (8% in Cohort II).

TABLE 3
General linear model for regression of BMI and
test factors on fasting insulin levels.

| | Cohort I | | Cohort II | |
|---|---|---|---|---|
| | F value | Pr > F | F value | Pr > F |
| Age (years) | 0.21 | 0.65 | 0.09 | 0.77 |
| Puberty (Tanner stages) | 0.20 | 0.66 | 0.32 | 0.57 |
| Sex | 1.39 | 0.24 | 1.10 | 0.31 |
| BMI (kg/m²) | 184.95 | 0.0001 | 76.7 | 0.0001 |
| HphI genotype | 5.24 | 0.0056 | 6.0 | 0.0033 |
| BMI*Sex | 14.51 | 0.0002 | 3.30 | 0.070 |
| BMI* HphI genotype | 22.76 | 0.0001 | 17.4 | 0.0001 |
| Sex* HphI genotype | 1.37 | 0.26 | 3.30 | 0.039 |
| BMI*Sex*HphI genotype | 1.17 | 0.31 | 0.23 | 0.79 |

Figure Legends

FIG. 2A consists of two graphs that demostrate the relationship between fasting plasma insulin and fatness in the 458 obese children of GenOb cohort I with respect to their HphI genotype. Patients were genotyped at the Hph1 locus as reported. The Hph1 [+/−] or [−/−] children were pooled as a single group since Hph1 [+/−] or [−/−] groups showed very similar regression and correlation coefficients. The regression between insulin and BMI in the Hph1 [+/+] patients (corresponding to VNTR I/II homozygotes) could be described by the linear equation Y=1.3X−20 (r=0.66, p<0.0001). Corresponding equations were Y=1.3X−21 (r=0.65) and Y=1.3X−22 (r=0.70), respectively, in the initial two cohorts of Mediterranean and Central Europe origin. The equation was Y=0.4X+3 (r=0.29, p<0.0001) in the Hph1 [+/−] or [−/−] obese children (corresponding to VNTR I/III or III/III genotypes), showing a lesser degree of correlation and a flatter slope. In the two initial cohorts, corresponding regression equations were very similar: Y=0.25X+7.5 (r=0.2) and Y=0.5X−1.6 (r=0.3). For a given degree of adiposity, children with HphI [+/−] or [−/−] genotypes had lower insulin values. One thousand randomizations were used to ascertain the significance of these genotypic effects (p<0.0001).

In the patients of replication Cohort II, the regression equation between insulin and BMI in the Hph1 [+/+] patients is Y=1.55X−28 (r=0.73, p<0.0001), while it is Y=0.16X+10.5 (r=0.14, p<0.23) in the [+/−] or [−/−] obese children (not shown).

FIG. 2B consists of two graphs that demonstrate the relationship between fasting plasma insulin and body mass index in the obese boys in the two Hph1 (insulin VNTR) genotype homozygous subgroups. The regression between insulin and BMI in the Hph1 [+/+] boys (corresponding to VNTR I/I homozygotes) fits the linear equation Y=1,5X−28 (r=0.74, p<0.0001). 55% of the variance in fasting insulin levels was explained by BMI in this genotypic subgroup. The equation is Y=0.5X+0 (r=0.31, p<0.005) in Hph1 [+/− or −/−] obese boys (corresponding to VNTR I/III or III/III genotypes) with less than 10% of insulin variance explained by BMI. For a given degree of adiposity, boys with the latter genotypes had less adjusted and lower insulin values.

In Cohort II the regression equation between insulin and BMI in the Hph1 [+/+] obese boys patients is Y=1.7X−32 (r=0.75, p<0.0001), while it is Y=0.35X+3.3 (r=0.09, p=0.08) in the [+/−] or [−/−] obese boys (not shown).

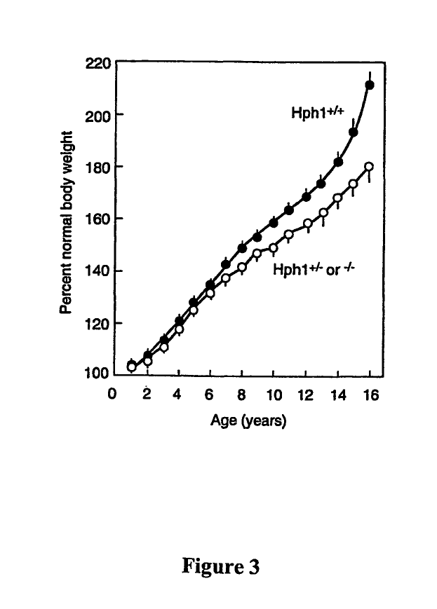

FIG. 3 is a graph that shows the averaged longitudinal weight curves (normalized to the normal weight value for age and height) versus chronological age in the two genotypic groups of obese children. The curves were constructed from the study of a subset of 332 patients whose yearly individual data could be collected from Health Personal Bulletins, starting at birth until time of study. Statistical analysis using repeated measures ANOVA as well as randomization-permutation tests indicated a highly significant difference between genotypic groups (p<0.001), the Hph1 [+/+] obese patients showing additional weight gain in late childhood.

### Example 4

### Evaluation of Type II Diabetes Risk in Obese Juveniles Carrying At Risk Genotypes

More than 70-80% of the young patients with obesity already have insulin resistance (IR) as detected using insulin clamp methodology, intravenous Glucose Tolerance tests with minimal model, and the HOMA insulin sensitivity index. This percentage is expected to increase with aging of patients and evolution of the obesity status.

Approximately 50% of young obese carry Ins Hph1 [+/−] or [−/−] genotypes (VNTR I/III and III/III). Approximately 80% of patients with those genotypes, according to the data described herein showed insufficient insulin secretion early in the evolution of obesity. They can therefore be expected to poorly match IR in the long term, which will progressively lead a fraction of them to Type II diabetes. In contrast, >80% of young obese with Is Hph1 [+/+] genotypes (VNTRI/I) are expected to be able to match IR and maintain long term euglycemia because of their abundant insulin secretion.

The estimated absolute risk of developing Type II diabetes is therefore about 80% in young obese with [+/−] or [−/−] Ins genotypes (VNTRI/III or III/III), and about 20% in the other genotypes. This corresponds to a four fold increase of the risk due to genotypic differences.

The data described herein for young obese show that early individual capacity for insulin secretion is dependent on BMI, insulin genotype and the interactive BMI * genotype. From this, is can be assumed that the risk of developing failure of insulin secretion and resulting Type II diabetes in the lifespan of young obese is also dependent on the same factors.

The estimation of about 80% risk of Type II diabetes in young obese carrying Hph1 [−] alleles (in homo or heterozygous states) is consistent with the observed prevalence of these genotypes in the general Type II diabetes population (see J. Todd, Ann Rev Genet (1996)), which is composed in equal parts of obese and non obese patients. This consistency relies on the assumption that the HphI [−] alleles (VNTR class III alleles) are diabetogenic only )or mostly) in obese patients, as suggested by the observation provided herein.

Studies are in progress to prove the hypothesis that HphI [−] alleles (VNTR III alleles) are predictive of Type II diabetes in patients with long-standing obesity or juvenile onset. VNTR genotypes are studied in two subgroups of adult obese patients with long-standing early onset obesity proven by photographs taken before the age of 25 years. Subgroup 1 will include obese adults with Type II diabetes; subgroup 2 will include obese adults comparable in all respects (sex distribution, age, duration of obesity, etc.) but who have maintained throughout the years near-normal blood glucose values. It is expected that an enrichment in [−] HphI alleles (VNTR class III alleles) will be observed in Subgroup 1 with Type II diabetes. The table below summarizes the expected results.

Estimated Risk of Diabetes with Regard to BMI and the Ins Genotypes

| | Relative | Relative Risk | | | |
|---|---|---|---|---|---|
| | Risk of Type | Depending on | | | |
| Adult BMI | II DM* | Hph1 Genotypes | | On VNTR Genotypes | |
| <21 | | +/+ | +/− or −/− | I/I | I/III or III/III |
| 25-27 | 2.0 | 1 | 2-4** | 1 | 2-4 |
| 29-31 | 6.2 | 2-3 | 9-11** | 2-3 | 9-11 |
| 33-35 | ˜10 | 3-5 | 12-20** | 3-5 | 12-20 |
| >35 | ˜25 | 8-12 | 35-50** | 8-12 | 35-50 |
| >40 | 40 | 10-20 | 45-80** | 10-20 | 45-80 |

*estimated from the literature (see Nokdad, et al., Diabetes Care 23: 1278-1283 (2000)), all genotypes confounded
**estimated assuming a 3-4.5 increase in RR in patients with [−] alleles (assumed from our results)

1. A method of determining the risk of developing non-insulin dependent diabetes mellitus (NIDDM) in an individual, comprising:
a) determining the identity of the polymorphic base(s) of at least one marker in linkage disquilibrium with the insulin HphI locus of said individual;
b) determining a body fat value for said individual; and
c) assigning a risk value based on said identity of step a), said body fat value of step b) and a predetermined value that correlates said identity, said body fat value and said risk of developing NIDDM.

2. A method of determining the risk of developing non-insulin dependent diabetes mellitus (NIDDM) in an individual, comprising:
a) determining the VNTR class of an insulin gene of said individual;
b) determining a body fat value for said individual; and
c) assigning a risk value based on said VNTR class of step a), said body fat value of step b) and a predetermined value that correlates said VNTR class, said body fat value and said risk of developing NIDDM.

3. A method of diagnosing a subtype of non-insulin dependent diabetes mellitus (NIDDM) in an individual, comprising:
a) determining the identity of the polymorphic base(s) of at least one marker in linkage disquilibrium with the insulin HphI locus of said individual;
b) determining a body fat value for said individual; and
c) assigning a subtype based on said identity of step a), said body fat value of step b) and a predetermined value that correlates said identity, said body fat value and likelihood of having a particular subtype of NIDDM.

4. A method of diagnosing a subtype of non-insulin dependent diabetes mellitus (NIDDM) in an individual, comprising:
a) determining the VNTR class of an insulin gene of said individual;
b) determining a body fat value for said individual; and
c) assigning a subtype based on said VNTR class of step a), said body fat value of step b) and a predetermined value that correlates said VNTR class, said body fat value and likelihood of having a particular subtype of NIDDM.

5. A method of treatment or prophylaxis of non-insulin dependent diabetes mellitus (NIDDM) for an individual, comprising:
a) a method of determining the risk of developing NIDDM according to either claim 1 or 2; and
b) administering a weight loss regime, wherein said weight loss regime is selected from the group consisting of food restriction, increased calorie use, gastrointestinal surgery, medicinal approaches and reduced absorption of dietary lipids.

6. A method of selecting an individual for inclusion in a clinical study or an association study that involves an insulin-related disorder, comprising:
a) determining the identity of the polymorphic base(s) of at least one marker in linkage disquilibrium with the insulin HphI locus of said individual;
b) determining a body fat value for said individual; and
c) including said individual in said study based on said identity of step a), said body fat value of step b) and a predetermined value that correlates said identity, said body fat value and said risk of developing an insulin-related disorder.

7. A method of selecting an individual for inclusion in a clinical study or an association study that involves an insulin-related disorder, comprising:
a) determining the VNTR class of an insulin gene of said individual;
b) determining a body fat value for said individual; and
c) including said individual in said study based on said VNTR class of step a), said body fat value of step b) and a predetermined value that correlates said VNTR class, said body fat value and said risk of developing an insulin-related disorder.

8. A method according to any one of claims 1, 3 or 6, wherein the identity of the polymorphic base(s) at said marker is determined for both copies of said marker present in said individual's genome.

9. A method according to any one of claims 2, 4 or 7, wherein the VNTR class of the insulin gene is determined for both copies of said VNTR present in said individual's genome.

10. A method of estimating the frequency of a haplotype for a set of genetic markers in a population suffering from juvenile obesity, comprising:
a) genotyping a marker in linkage disquilibrium with th insulin HphI locus by determining the identity of the nucleotides at said marker for both copies of said marker present in the genome of each individual in said population;
b) genotyping a second marker by determining the identity of the nucleotides at said second genetic marker for both copies of said second marker present in the genome of each individual in said population; and
c) applying a haplotype determination method to the identities of the nucleotides determined in steps a) and b) to obtain an estimate of said frequency.

11. A method according to claim 10, wherein said haplotype determination method is selected from the group consisting of asymmetric PCR amplification, double PCR amplification of specific alleles, the Clark method, or an expectation maximization algorithm.

12. A method of determining the risk of developing non-insulin dependent diabetes mellitus (NIDDM) in an individual, comprising:
a) genotyping a marker in linkage disquilibrium with the insulin HphI locus by determining the identity of the nucleotides at said marker for both copies of said marker present in the genome of an individual;
b) determining a body fat value for said individual; and
c) assigning a risk value based on said identity of step a), said body fat value of step c) and a predetermined value that correlates said identity, said body fat value and said risk of developing NIDDM.

13. A method of diagnosing a subtype of non-insulin dependent diabetes mellitus (NIDDM) in an individual, comprising:
a) genotyping a marker in linkage disquilibrium with the insulin HphI locus by determining the identity of the nucleotides at said marker for both copies of said marker present in the genome of an individual;
b) determining a body fat value for said individual; and
c) assigning a subtype based on said identity of step a), said body fat value of step b) and a predetermined value that correlates said identity, said body fat value and likelihood of having a particular subtype of NIDDM.

14. A method of selecting an individual for inclusion in a clinical study or an association study that involves an insulin-related disorder, comprising:
a) genotyping a marker in linkage disquilibrium with the insulin HphI locus by determining the identity of the nucleotides at said marker for both copies of said marker present in the genome of an individual;
b) determining a body fat value for said individual; and
c) including said individual in said study based on said identity of step a), said body fat value of step b) and a predetermined value that correlates said identity, said body fat value and risk of developing an insulin-related disorder.

15. A method according to any one of claims 10, 12, 13 or 14, wherein said second marker is in linkage disquilibrium with the insulin HphI locus.

16. A method of detecting an association between a haplotype and an insulin-related disorder, comprising:
a) estimating the frequency of at least one haplotype in a population suffering from said insulin-related disorder according to the method of claim 10;
b) estimating the frequency of said haplotype in a control population according to the method of claim 10; and
c) determining whether a statistically significant association exists between said haplotype and said insulin-related disorder.

17. A method according to any one of claims 6, 7 or 16, wherein said insulin-related disorder is hyperinsulinemia or a predisposition to hyperinsulinemia.

18. A method according to any one of claims 1, 3, 6, 10, 12, 13 or 14, wherein said marker in linkage disquilibrium with the insulin HphI locus is selected from the group consisting of the markers described in Table C.

19. A method according to any one of claims 1, 3, 6, 10, 12, 13 or 14, wherein said marker in linkage disquilibrium with the insulin HphI locus is selected from the group consisting of −4217 PstI, −2221 MspI, −23 HphI, +1428 FokI, +11000 AluI and +32000 ApaI.

20. A method according to any one of claims 1, 3, 6, 10, 12, 13 or 14, wh rein said marker in linkage disquilibrium with th insulin HphI locus is −23 HphI.


SEQUENCE LISTING (12 sequences)

SEQ ID NO: 1 - DNA, 20 bp, Artificial Sequence
  Primer
tgacgccaaggacaagctca

SEQ ID NO: 2 - DNA, 20 bp, Artificial Sequence
  Primer
ccagcagccccagtcctgca

SEQ ID NO: 3 - DNA, 20 bp, Artificial Sequence
  Primer
caccagctggccttcaaggt

SEQ ID NO: 4 - DNA, 20 bp, Artificial Sequence
  Primer
gctgggcactaacaaggtgt

SEQ ID NO: 5 - DNA, 20 bp, Artificial Sequence
  Primer
tccaggacaggctgcatcag

SEQ ID NO: 6 - DNA, 20 bp, Artificial Sequence
  Primer
agcaatgggcggttggctca

SEQ ID NO: 7 - DNA, 18 bp, Artificial Sequence
  Primer
taaagcccttgaaccagc

SEQ ID NO: 8 - DNA, 23 bp, Artificial Sequence
  Primer
cagcccagcctcctccctccaca

SEQ ID NO: 9 - DNA, 19 bp, Artificial Sequence
  Primer
cccaggggccgaagagtca

SEQ ID NO: 10 - DNA, 20 bp, Artificial Sequence
  Primer
gctgagctggcagcgattca

SEQ ID NO: 11 - DNA, 22 bp, Artificial Sequence
  Primer
cttggactttgagtcaaattgg

SEQ ID NO: 12 - DNA, 20 bp, Artificial Sequence
  Primer
cctcctttggtcttactggg